(19) United States
(12) Patent Application Publication
(10) Pub. No.: US 2021/0395831 A1
(43) Pub. Date: Dec. 23, 2021

(54) BIOMARKERS TO IMPROVE EFFICACY OF CANCER IMMUNOTHERAPY

(71) Applicant: Arch Oncology, Inc., Brisbane, CA (US)

(72) Inventors: Benjamin J. CAPOCCIA, Webster Groves, MO (US); Robyn PURO, St. Louis, MO (US); W. Casey WILSON, St. Louis, MO (US); Pamela T. MANNING, Chesterfield, MO (US); Robert W. KARR, Frontenac, MO (US)

(21) Appl. No.: 17/292,121

(22) PCT Filed: Nov. 8, 2019
(86) PCT No.: PCT/US2019/060620

§ 371 Date: May 7, 2021

Related U.S. Application Data
(60) Provisional application No. 62/758,368, filed on Nov. 9, 2018.

(51) Int. Cl. C12Q 1/6886; C07K 16/28; G01N 33/50

(52) U.S. Cl. CPC C12Q 1/6886; C07K 16/2803; C12Q 2600/158; C12Q 2600/106; G01N 33/5011

(57) ABSTRACT

Provided are methods for treatment of cancer. Also provided are methods for treating a patient susceptible, or suspected of being susceptible, with anti-CD47 therapy.

This application claims the benefit of priority of U.S. Provisional Application No. 62/758,368, filed Nov. 9, 2018, the disclosure of which is hereby incorporated by reference as if written herein in its entirety.

### BACKGROUND OF THE DISCLOSURE

The development of cancer immunotherapies is occurring at a rapid pace. These immunotherapy treatments enhance the cytotoxic activity of cells of the immune system and have resulted in improved survival of patients with tumor types as diverse as melanoma, non-small cell lung cancer, bladder cancer, and Hodgkin's lymphoma. Despite these positive results, there remains a significant patient population that fail to respond to prescribed immunotherapy treatment or respond initially only to eventually acquire resistance.

The identification and use of biomarkers in the clinic would significantly improve the use of these immunotherapies. Not only would health care providers be able to identify patients that are most likely to benefit from these therapies, biomarkers may avoid treatment-related toxicity and increase our understanding of modes of action of immunotherapy and thereby identify potential combination therapies.

Furthermore, discovering and validating new biomarkers remains an extremely active area of research and development, especially with respect to: other types of immune cells, the complexity of tumor-immune interactions, and the steps involved in the process of the immune system launching an adaptive response against tumors. Thus, there remains a need for advances in biomarker development to further understand the relationship between cancer, the immune system, and the efficacy of immunotherapies.

### DETAILED DESCRIPTION OF THE DRAWINGS

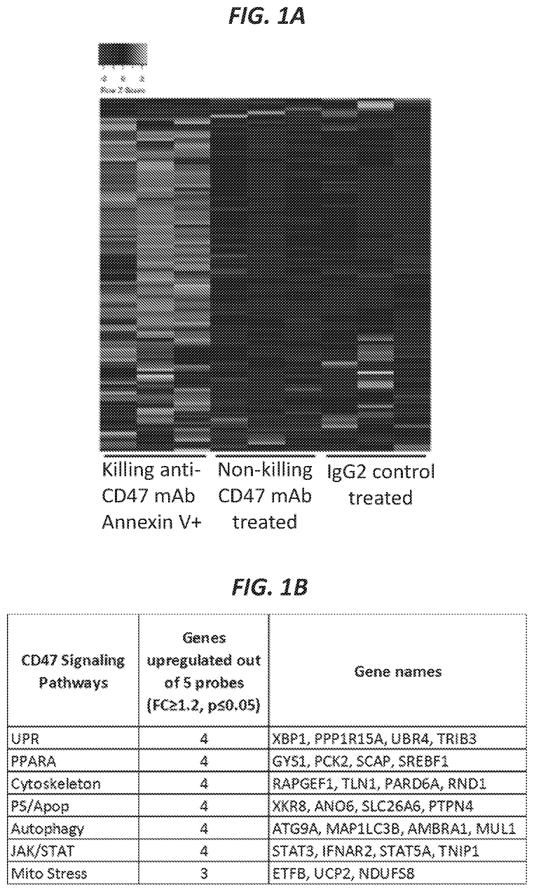

FIG. 1A-FIG. 1B. Treatment with an anti-CD47 antibody induces molecular patterns of PCDIII and ICD. FIG. 1A. Gene expression heat map from Annexin V⁺ Jurkat human leukemia cells treated with an anti-CD47 Ab which induces cell death compared to treated cells with an anti-CD47 Ab that does not induce cell death or IgG2 Ab (control) treated cells. FIG. 1B. Pathway enrichment analysis of genes upregulated in Annexin V+ cells treated with an anti-CD47 Ab which induces cell death compared to treated cells with an IgG2 Ab (control).

### DETAILED DESCRIPTION

Disclosed herein are methods for treating cancer in a patient and for identifying a patient likely to respond to treatment with an agent that specifically binds to CD47, wherein the agent is an anti-CD47 antibody or antigen binding fragment thereof. The methods disclosed herein use multiple assays of biomarkers contained with a biological sample obtained from a patient. In certain embodiments, diagnosing and treating a cancer in a patient who has not received therapy, comprises obtaining a first biological sample from a patient and administering an anti-CD47 antibody to the biological sample in vitro;

quantifying the amount of at least one biomarker in:
i. a biological sample treated with an anti-CD47 antibody or antigen binding fragment thereof; and
ii. in an untreated biological sample, wherein the at least one biomarker is selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof, and comparing the amounts of the at least one biomarker in an treated biological sample with the biomarker in the untreated biological sample; identifying a patient as responsive to anti-CD47 therapy when the amount of at least one biomarker in the treated biological sample is greater than the amount of at least one biomarker in the untreated biological sample from the patient; and

administering a therapeutically effective amount of an anti-CD47 antibody or antigen binding fragment thereof to the patient.

In certain embodiments, the method of quantifying the amount of the biomarker in each sample is not limited to one or more methods selected from NanoString gene expression profiling, RNAseq, qPCR, and microarray.

In certain embodiments, the anti-CD47 antibody or antigen binding fragment thereof can be used (alone or in combination with other therapeutic agents or procedures) to treat, prevent and/or diagnose disorders, including immune disorders and cancer.

In certain embodiments, the cancer is a solid tumor, leukemia, a lymphoma, multiple myeloma.

In certain embodiments, the solid tumor is selected from ovarian cancer, breast cancer, endometrial cancer, colon cancer (colorectal cancer), rectal cancer, bladder cancer, urothelial cancer, lung cancer (non-small cell lung cancer, adenocarcinoma of the lung, squamous cell carcinoma of the lung), bronchial cancer, bone cancer, prostate cancer, pancreatic cancer, gastric cancer, adrenocortical carcinoma, hepatocellular carcinoma, adult (primary) liver cancer, gall bladder cancer, bile duct cancer, esophageal cancer, renal cell carcinoma, thyroid cancer, squamous cell carcinoma of the head and neck (head and neck cancer), testicular cancer, cancer of the endocrine gland, cancer of the adrenal gland, cancer of the pituitary gland, cancer of the skin, cancer of soft tissues, cancer of blood vessels, cancer of brain, cancer of nerves, cancer of eyes, cancer of meninges, cancer of oropharynx, cancer of hypopharynx, cancer of cervix, and cancer of uterus, glioblastoma, meduloblastoma, astrocytoma, glioma, meningioma, gastrinoma, neuroblastoma, melanoma, and myelodysplastic syndrome.

In certain embodiments, the leukemia is selected from systemic mastocytosis, acute lymphocytic (lymphoblastic) leukemia (ALL), T-cell—ALL, acute myeloid leukemia (AML), chronic lymphocytic leukemia (CLL), chronic myeloid leukemia (CML), myeloproliferative disorder/neoplasm, myelodysplastic syndrome, monocytic cell leukemia, and plasma cell leukemia.

In certain embodiments, the lymphoma is selected from histiocytic lymphoma and T-cell lymphoma, B cell lymphomas, including Hodgkin's lymphoma and non-Hodgkin's lymphoma, such as low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, and Waldenstrom's Macroglobulinemia.

In certain embodiments, the sarcoma is selected from osteosarcoma, Ewing's sarcoma, leiomyosarcoma, synovial sarcoma, alveolar soft part sarcoma, angiosarcoma, liposarcoma, fibrosarcoma, rhabdomyosarcoma, and chrondrosarcoma. In certain embodiments, the biological sample is selected from, but not limited to, a core biopsy, a free needle aspirate, a pleural effusion, a resection, ascites, whole blood, blood serum, plasma, bone marrow, or other bodily fluid, or dilution thereof.

In certain embodiments the least two biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

In certain embodiments, the at least three biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

In certain embodiments, the at least four biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

In certain embodiments, the at least five biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

In certain embodiments, greater that five biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

In certain embodiments, the baseline standard is a unit of measurement which provides a calibrated level of the biological effect which may occur prior to the administration of a therapy; i.e. an anti-CD47 antibody. As described herein, you may achieve a baseline standard by measuring gene expression levels for the one or more biomarkers described herein, in untreated tumor cells; i.e., without the administration of an anti-CD47 antibody or antigen binding fragment thereof to establish a baseline standard for treated tumor cells; i.e., with the administration of an anti-CD47 antibody when measured in the same patient.

In certain embodiments, the baseline standard is established for different tumor cell types; i.e., solid tumors and hematological tumors in an untreated patient when measured in the same patient.

In certain embodiments, the anti-CD47 antibodies or antigen binding fragments thereof disclosed block the CD47/SIRPα interaction, reversing the ‘don't eat me’ signal.

In certain embodiments, the anti-CD47 antibodies or antigen binding fragments thereof disclosed induce cell death in solid and hematopoietic cell tumor lines.

In certain embodiments, the anti-CD47 antibodies or antigen binding fragments thereof disclosed have tumor cell binding selectivity compared to normal cells, particularly, binding negligibly to red blood cells (RBCs) in contrast to tumor cells even at high concentrations of antibody.

In certain embodiments, the anti-CD47 antibodies or antigen binding fragments thereof disclosed comprise a combination of a heavy chain (HC) and a light chain (LC), wherein the combination is selected from:

a heavy chain comprising the amino acid sequence of SEQ ID NO:1 and a light chain comprising the amino acid sequence SEQ ID NO:2;

a heavy chain comprising the amino acid sequence of SEQ ID NO:3 and a light chain comprising the amino acid sequence SEQ ID NO:4;

a heavy chain comprising the amino acid sequence of SEQ ID NO:5 and a light chain comprising the amino acid sequence SEQ ID NO:6;

a heavy chain comprising the amino acid sequence of SEQ ID NO:7 and a light chain comprising the amino acid sequence SEQ ID NO:6;

a heavy chain comprising the amino acid sequence of SEQ ID NO:8 and a light chain comprising the amino acid sequence SEQ ID NO:9; and

a heavy chain comprising the amino acid sequence of SEQ ID NO:7 and a light chain comprising the amino acid sequence SEQ ID NO:10.

Summary of Sequences

| SEQ | | |
|---|---|---|
| ID | | |
| NO: | Description | Sequence |
| 1 | Vx4humH01 | QVQLVQSGAEVKKPGASVQVSCKASGYTFTNYVIHWLRQAPGQGLEWMGYIYPYNDGILYNE |
| | Full Length | KFKGRVTMTSDTSISTAYMELSSLRSDDTAVYYCARGGYYVPDYWGQATLVTVSSASTKGPSV |
| | HC | FPLAPCSRSTSESTAALGCLVKDYFPEPVTVSWNSGALTSGVHTFPAVLQSSGLYSLSSVVTVPS |
| | | SSLGTKTYTCNVDHKPSNTKVDKRVESKYGPPCPPCPAPEFEGGPSVFLEPPKPKDTLMISRTPE |
| | | VTCVVVDVSQEDPEVQFNWYVDGVEVHNAKTKPREEQFNSTYRVVSVLTVLHQDWLNGKEY |
| | | KCKVSNKGLPSSIEKTISKAKGQPREPQVYTLPPSQEEMTKNQVSLTCLVKGFYPSDIAVEWESN |
| | | GQPENNYKTTPPVLDSDGSFFLYSRLTVDKSRWQEGNVFSCSVMHEALHNHYTQKSLSLSLGK |
| 2 | Vx4humL01 | DIVMTQSPLSLPVTPGEPASISCRSRQSIVHTNGNTYLGWYLQKPGQSPRLLIYKVSNRFSGVPDR |
| | Full Length | FSGSGSGTDFTLKISRVEADDVGIYYCFQGSHVPYTFGQGTKLEIKRTVAAPSVFIFPPSDEQLKS |
| | LC | GTASVVCLLNNFYPREAKVQWKVDNALQSGNSQESVTEQDSKDSTYSLSSTLTLSKADYEKHK |
| | | VYACEVTHQGLSSPVTKSFNRGEC |
| 3 | Vx8humH11 | QVQLVQSGAEVKKPGASVQVSCKASGYSFTNYYIHWLRQAPGQGLEWMGYIDPLNGDTTYNQ |
| | Full Length | KFKGRVTMTSDTSISTAYMELSSLRSDDTAVYYCARGGKRAMDYWGQATLVTVSSASTKGPS |
| | HC | VFPLAPCSRSTSESTAALGCLVKDYFPEPVTVSWNSGALTSGVHTFPAVLQSSGLYSLSSVVTVP |
| | | SSSLGTKTYTCNVDHKPSNTKVDKRVESKYGPPCPPCPAPEFEGGPSVFLFPPKPKDTLMISRTPE |
| | | VTCVVVDVSQEDPEVQFNWYVDGVEVHNAKTKPREEQFNSTYRVVSVLTVLHQDWLNGKEY |
| | | KCKVSNKGLPSSIEKTISKAKGQPREPQVYTLPPSQEEMTKNQVSLTCLVKGFYPSDIAVEWESN |
| | | GQPENNYKTTPPVLDSDGSFFLYSRLTVDKSRWQEGNVFSCSVMHEALHNHYTQKSLSLSLGK |
| 4 | Vx8humL03 | DIVMTQSPLSLPVTPGEPASISCRASQDISNYLNWYLQKPGQSPRLLIYYTSRLYSGVPDRFSGSG |
| | Full Length | SGTDFTLKISRVEADDVGIYYCQQGNTLPWTFGQGTKLEIKRTVAAPSVFIFPPSDEQLKSGTAS |
| | LC | VVCLLNNFYPREAKVQWKVDNALQSGNSQESVTEQDSKDSTYSLSSTLTLSKADYEKHKVYAC |
| | | EVTHQGLSSPVTKSFNRGEC |
| 5 | Vx9humH12 | QVQLVQSGAEVKKPGASVKVSCKASGYTFTNYWIHWVRQAPGQGLEWMGYTDPRTDYTEYN |
| | Full Length | QKFKDRVTMTRDTSTSTVYMELSSLRSEDTAVYYCARGGRVGLGYWGQGTLVTVSSASTKGP |
| | HC | SVFPLAPCSRSTSESTAALGCLVKDYFPEPVTVSWNSGALTSGVHTFPAVLQSSGLYSLSSVVTV |
| | | PSSNFGTQTYTCNVDHKPSNTKVDKTVERKCCVECPPCPAPPVAGPSVFLFPPKPKDTLMISRTP |
| | | EVTCVVVDVSHEDPEVQFNWYVDGVEVHNAKTKPREEQFNSTFRVVSVLTVVHQDWLNGKE |
| | | YKCKVSNKGLPAPIEKTISKTKGQPREPQVYTLPPSREEMTKNQVSLTCLVKGFYPSDIAVEWES |
| | | NGQPENNYKTTPPMLDSDGSFFLYSKLTVDKSRWQQGNVFSCSVMHEALHNHYTQKSLSLSPG |
| | | K |
| 6 | Vx9humL02 | DIVMTQSPDSLAVSLGERATINCRSSQNIVQSNGNTYLEWYQQKPGQPPKLLIYKVFHRFSGVPD |
| | Full Length | RFSGSGSGTDFTLTISSLQAEDVAVYYCFQGSHVPYTFGQGTKLEIKRTVAAPSVFIFPPSDEQLK |
| | LC | SGTASVVCLLNNFYPREAKVQWKVDNALQSGNSQESVTEQDSKDSTYSLSSTLTLSKADYEKH |
| | | KVYACEVTHQGLSSPVTKSFNRGEC |
| 7 | Vx9humH14 | EVQLVQSGAEVKKPGESLKISCKGSGYTFTNYWIHWVRQMPGKGLEWMGYTDPRTDYTEYNQ |
| | Full Length | KFKDQVTISADKSISTAYLQWSSLKASDTAMYYCARGGRVGLGYWGQGTLVTVSSASTKGPSV |
| | HC | FPLAPCSRSTSESTAALGCLVKDYFPEPVTVSWNSGALTSGVHTFPAVLQSSGLYSLSSVVTVPS |
| | | SNFGTQTYTCNVDHKPSNTKVDKTVERKCCVECPPCPAPPVAGPSVFLFPPKPKDTLMISRTPEV |
| | | TCVVVDVSHEDPEVQFNWYVDGVEVHNAKTKPREEQFNSTERVVSVLTVVHQDWLNGKEYK |
| | | CKVSNKGLPAPIEKTISKTKGQPREPQVYTLPPSREEMTKNQVSLTCLVKGFYPSDIAVEWESNG |
| | | QPENNYKTTPPMLDSDGSFFLYSKLTVDKSRWQQGNVFSCSVMHEALHNHYTQKSLSLSPGK |
| 8 | Vx4humH05 | QVQLVQSGAEVKKPGASVKVSCKASGYTFTNYVIHWVRQAPGQGLEWMGYIYPYNDGILYNE |
| | Full Length | KFKGRVTMTTDTSTSTAYMELRSLRSDDTAVYYCARGGYYVPDYWGQGTTVTVSSASTKGPS |
| | HC | VFPLAPCSRSTSESTAALGCLVKDYFPEPVTVSWNSGALTSGVHTFPAVLQSSGLYSLSSVVTVP |
| | | SSSLGTKTYTCNVDHKPSNTKVDKRVESKYGPPCPPCPAPEFEGGPSVFLFPPKPKDTLMISRTPE |
| | | VTCVVVDVSQEDPEVQFNWYVDGVEVHNAKTKPREEQFNSTYRVVSVLTVLHQDWLNGKEY |
| | | KCKVSNKGLPSSIEKTISKAKGQPREPQVYTLPPSQEEMTKNQVSLTCLVKGFYPSDIAVEWESN |
| | | GQPENNYKTTPPVLDSDGSFFLYSRLTVDKSRWQEGNVFSCSVMHEALHNHYTQKSLSLSLGK |
| 9 | Vx4humL02 | DVVMTQSPLSLPVTLGQPASISCRSRQSIVHTNGNTYLGWFQQRPGQSPRRLIYKVSNRFSGVPD |
| | Full Length | RFSGSGSGTDFTLKISRVEAEDVGVYYCFQGSHVPYTFGQGTKLEIKRTVAAPSVFIFPPSDEQLK |
| | LC | SGTASVVCLLNNFYPREAKVQWKVDNALQSGNSQESVTEQDSKDSTYSLSSTLTLSKADYEKH |
| | | KVYACEVTHQGLSSPVTKSFNRGEC |
| 10 | Vx9humL07 | DVVMTQSPLSLPVTLGQPASISCRSSQNIVQSNGNTYLEWFQQRPGQSPRRLIYKVFHRFSGVPD |
| | Full Length | RFSGSGSGTDFTLKISRVEAEDVGVYYCFQGSHVPYTFGQGTKLEIKRTVAAPSVFIFPPSDEQLK |
| | LC | SGTASVVCLLNNFYPREAKVQWKVDNALQSGNSQESVTEQDSKDSTYSLSSTLTLSKADYEKH |
| | | KVYACEVTHQGLSSPVTKSFNRGEC |
| 11 | Homo | GCTATGGTGGTGGTGGCAGCCGCGCCGAACCCGGCCGACGGGACCCCTAAAGTTCTGCTTC |
| | sapiens | TGTCGGGGCAGCCCGCCTCCGCCGCCGGAGCCCCGGCCGGCCAGGCCCTGCCGCTCATGGT |
| | XBP1 full | GCCAGCCCAGAGAGGGGCCAGCCCGGAGGCAGCGAGCGGGGGGCTGCCCCAGGCGCGCAA |
| | length open | GCGACAGCGCCTCACGCACCTGAGCCCCGAGGAGAAGGCGCTGAGGAGGAAACTGAAAAA |
| | reading | CAGAGTAGCAGCTCAGACTGCCAGAGATCGAAAGAAGGCTCGAATGAGTGAGCTGGAACA |
| | frame (ORF) | GCAAGTGGTAGATTTAGAAGAAGAGAACCAAAAACTTTTGCTAGAAAATCAGCTTTTACGA |
| | cDNA clone. | GAGAAAACTCATGGCCTTGTAGTTGAGAACCAGGAGTTAAGACAGCGCTTGGGGATGGATG |
| | GenBank: | CCCTGGTTGCTGAAGAGGAGGCGGAAGCCAAGGGGAATGAAGTGAGGCCAGTGGCCGGGT |
| | CR456611.1 | CTGCTGAGTCCGCAGCACTCAGACTACGTGCACCTCTGCAGCAGGTGCAGGCCCAGTTGTC |
| | | ACCCCTCCAGAACATCTCCCCATGGATTCTGGCGGTATTGACTCTTCAGATTCAGAGTCTGA |
| | | TATCCTGTTGGGCATTCTGGACAACTTGGACCCAGTCATGTTCTTCAAATGCCCTTCCCCAG |
| | | AGCCTGCCAGCCTGGAGGAGCTCCCAGAGGTCTACCCAGAAGGACCCAGTTCCTTACCAGC |
| | | CTCCCTTTCTCTGTCAGTGGGGACGTCATCAGCCAAGCTGGAAGCCATTAATGAACTAATTC |
| | | GTTTTGACCACATATATACCAAGCCCCTAGTCTTAGAGATACCCTCTGAGACAGAGAGCCA |
| | | AGCTAATGTGG |
| 12 | Homo | ATGGCCCCAGGCCAAGCACCCCATCAGGCTACCCCGTGGAGGGATGCCCACCCTTTCTTCCT |
| | sapiens full | CCTGTCCCCAGTGATGGGCCTCCTCAGCCGCACCTGGAGCCGCCTGAGGGGCCTGGGACCT |
| | open reading | CTAGAGCCCTGGCTGGTGGAAGCAGTAAAAGGAGCAGCTCTGGTAGAAGCTGGCCTGGAG |
| | frame cDNA | GGAGAAGCTAGGACTCCTCTGGCAATCCCCCATACCCCTTGGGGCAGACGCCCTGAAGAGG |
| | clone | AGGCTGAAGACAGTGGAGGCCCTGGAGAGGACAGAGAAACACTGGGGCTGAAAACCAGCA |
| | RZPDo834C | GTTCCCTTCCTGAAGCCTGGGGACTTTTGGATGATGATGATGGCATGTATGGTGAGCGAGAG |
| | 036D for | GCAACCAGTGTCCCTAGAGGGCAGGGAAGTCAATTTGCAGATGGCCAGCGTGCTCCCCTGT |
| | gene | CTCCCAGCCTTCTGATAAGGACACTGCAAGGTTCTGATAAGAACCCAGGGGAGGAGAAAGC |
| | PPP1R15A, | CGAGGAAGAGGGAGTTGCTGAAGAGGAGGGAGTTAACAAGTTCTCTTATCCACCATCACAC |
| | protein. | CGGGAGTGTTGTCCAGCCGTGGAGGAGGAGGACGATGAAGAAGCTGTAAAGAAAGAAGCT |
| | phosphatase | CACAGAACCTCTACTTCTGCCTTGTCTCCAGGATCCAAGCCCAGCACTTGGGTGTCTTGCCC |
| | 1, regulatory | AGGGGAGGAAGAGAATCAAGCCACGGAGGATAAAAGAACAGAAAGAAGTAAAGGAGCCA |
| | (inhibitor) | GGAAGACCTCCGTGTCCCCCCGATCTTCAGGCTCCGACCCCAGGTCCTGGGAGTATCGTTCA |
| | subunit 15A. | GGAGAGGCGTCCGAGGAGAAGGAGGAAAAGGCACACAAAGAAACTGGGAAAGGAGAAGC |
| | GenBank: | TGCCCCAGGGCCGCAATCCTCAGCCCCAGCCCAGAGGCTCCAGCTCAAGTCCTGGTGGTGC |
| | CR457259.1 | CAACCCAGTGATGAAGAGGAGGGTGAGGTCAAGGCTTTGGGGGCAGCTGAGAAGGATGGA |
| | | GAAGCTGAGTGTCCTCCCTGCATCCCCCCACCAAGTGCCTTCCTGAAGGCCTGGGTGTATTG |
| | | GCCAGGAGAGGACACAGAGGAAGAGGAAGATGAGGAAGAAGATGAGGACAGTGACTCTG |
| | | GATCAGATGAGGAAGAGGGAGAAGCTGAGGCTTCCTCTTCCACTCCTGCTACAGGTGTCTT |
| | | CTTGAAGTCCTGGGTCTATCAGCCAGGAGAGGACACAGAGGAGGAGGAAGATGAGGACAG |
| | | TGATACAGGATCAGCCGAGGATGAAAGAGAAGCTGAGACTTCTGCTTCCACACCCCCTGCA |
| | | AGTGCTTTCTTGAAGGCCTGGGTGTATCGGCCAGGAGAGGACACGGAGGAGGAGGAAGAT |
| | | GAGGATGTGGATAGTGAGGATAAGGAAGATGATTCAGAAGCAGCCTTGGGAGAAGCTGAG |
| | | TCAGACCCACATCCCTCCCACCCGGACCAGAGGGCCCACTTCAGGGGCTGGGGATATCGAC |
| | | CTGGAAAAGAGACAGAGGAAGAGGAAGCTGCTGAGGACTGGGGAGAAGCTGAGCCCTGCC |
| | | CCTTCCGAGTGGCCATCTATGTACCTGGAGAGAAGCCACCGCCTCCCTGGGCTCCTCCTAGG |
| | | CTGCCCCTCCGACTGCAAAGGCGGCTCAAGCGCCCAGAAACCCCTACTCATGATCCGGACC |
| | | CTGAGACTCCCCTAAAGGCCAGAAAGGTGCGCTTCTCCGAGAAGGTCACTGTCCATTTCCTG |
| | | GCTGTCTGGGCAGGGCCGGCCCAGGCCGCCCGCCAGGGCCCCTGGGAGCAGCTTGCTCGGG |
| | | ATCGCAGCCGCTTCGCACGCCGCATCACCCAGGCCCAGGAGGAGCTGAGCCCCTGCCTCAC |
| | | CCCTGCTGCCCGGGCCAGAGCCTGGGCACGCCTCAGGAACCCACCTTTAGCCCCCATCCCTG |
| | | CCCTCACCCAGACCTTGCCTTCCTCCTCTGTCCCTTCGTCCCCAGTCCAGACCACGCCCTTGA |
| | | GCCAAGCTGTGGCCACACCTTCCCGCTCGTCTGCTGCTGCAGCGGCTGCCCTGGACCTCAGT |
| | | GGGAGGCGTGGTTAA |
| 13 | Homo | GAAGGCCCTGGGCACCCTGGGCATGACGACAAATGAAAAGGGCCAGGTCGTGACCAAGAC |
| | sapiens | AGCACTCCTGAAGCAGATGGAAGAGCTGATCGAGGAGCCTGGCCTCACGTGCTGCATCTGC |
| | ubiquitin | AGGGAGGGATACAAGTTCCAGCCCACAAAGGTCCTGGGCATTTATACCTTCACGAAGCGGG |
| | protein | TAGCCTTGGAGGAGATGGAGAATAAGCCCCGGAAACAGCAGGGCTACAGCACCGTGTCCC |
| | ligase E3 | ACTTCAACATTGTGCACTACGACTGCCATCTGGCTGCCGTCAGGTTGGCTCGAGGCCGGGAA |
| | component | GAGTGGGAGAGTGCCGCCCTGCAGAATGCCAACACCAAGTGCAACGGGCTCCTTCCGGTCT |
| | n-recognin | GGGGACCTCATGTCCCTGAATCAGCTTTTGCCACTTGCTTGGCAAGACACAACACTTACCTC |
| | 4, mRNA. | CAGGAATGTACAGGCCAGCGGGAGCCCACGTATCAGCTCAACATCCATGACATCAAACTGC |
| | GenBank: | TCTTCCTGCGCTTCGCCATGGAGCAGTCGTTCAGCGCAGACACTGGCGGGGGCGGCCGGGA |
| | BC007962.2. | GAGCAACATCCACCTGATCCCGTACATCATTCACACTGTGCTTTACGTCCTGAACACAACCC |
| | | GAGCAACTTCCCGAGAAGAGAAGAACCTCCAAGGCTTTCTGGAACAGCCCAAGGAGAAGT |
| | | GGGTGGAGAGTGCCTTTGAAGTGGACGGGCCCTACTATTTCACAGTCTTGGCCCTTCACATC |
| | | CTGCCCCCTGAGCAGTGGAGAGCCACACGTGTGGAAATCTTGCGGAGGCTGTTGGTGACCT |
| | | CGCAGGCTCGGGCAGTGGCTCCAGGTGGAGCCACCAGGCTGACAGATAAGGCAGTGAAGG |
| | | ACTATTCCGCTTACCGTTCTTCCCTTCTCTTTTGGGCCCTCGTCGATCTCATTTACAACATGTT |
| | | TAAGAAGGTGCCTACCAGTAACACAGAGGGAGGCTGGTCCTGCTCTCTCGCTGAGTACATC |
| | | CGCCACAACGACATGCCCATCTACGAAGCTGCCGACAAAGCCCTGAAAACCTTCCAGGAGG |
| | | AGTTCATGCCAGTGGAGACCTTCTCAGAGTTCCTCGATGTGGCCGGTCTTTTATCAGAAATC |
| | | ACCGATCCAGAGAGCTTCCTGAAGGACCTGTTGAACTCAGTCCCCTGACCACCACACAGCA |
| | | GCTGCGGCGGCGAAGACGAAGCTGGCTTGCCTTCCACCCTCTGTTCTCCCTCCTTGTGCATT |
| | | AAGTTCCCTCCGCGGGATGCTGCATTGTTACCCCGCCCTCCCCTCTCTCATTTTTCTTGGTGT |
| | | GGCTTGGGGTTTTTAGGCTTCCTGTTTTATCTCGTGTGTGTGGTGCACCAGCTATGAGGTTGT |
| | | CTGTAACCCAAGCCATCAAAGGGCCTGTACATACCTAGGAGCCATGAGTTGTCCCGGCCAG |
| | | CTTCATACTTGAGTGTGCACATCTTGAGAAATAAACAAGTGACTTAACACAAAAAAAAAAA |
| | | AAAAAAA |
| 14 | Homo | GTTCGTTGCAACAAATTGATGAGCAATGCTTTTTTATAATGCCAACTTTGTACAAAAAAGTT |
| | sapiens clone | GGCATGCGAGCCACCCCTCTGGCTGCTCCTGCGGGTTCCCTGTCCAGGAAGAAGCGGTTGG |
| | CCSBHm_00010895 | AGTTGGATGACAACTTAGATACCGAGCGTCCCGTCCAGAAACGAGCTCGAAGTGGGCCCCA |
| | TRIB3 | GCCCAGACTGCCCCCCTGCCTGTTGCCCCTGAGCCCACCTACTGCTCCAGATCGTGCAACTG |
| | (TRIB3) | CTGTGGCCACTGCCTCCCGTCTTGGGCCCTATGTCCTCCTGGAGCCCGAGGAGGGCGGGCGG |
| | mRNA. | GCCTACCGGGCCCTGCACTGCCCTACAGGCACTGAGTATACCTGCAAGGTGTACCCCGTCC |
| | GenBank: | AGGAAGCCCTGGCCGTGCTGGAGCCCTATGCGCGGCTGCCCCCGCACAAGCATGTGGCTCG |
| | KR710261.1 | GCCCACTGAGGTCCTGGCTGGTACCCAGCTCCTCTACGCCTTTTTCACTCGGACCCATGGGG |
| | | ACATGCACAGCCTGGTGCGAAGCCGCCACCGTATCCCTGAGCCTGAGGCTGCCGTGCTCTTC |
| | | CGCCAGATGGCCACCGCCCTGGCGCACTGTCACCAGCACGGTCTGGTCCTGCGTGATCTCA |
| | | AGCTGTGTCGCTTTGTCTTCGCTGACCGTGAGAGGAAGAAGCTGGTGCTGGAGAACCTGGA |
| | | GGACTCCTGCGTGCTGACTGGGCCAGATGATTCCCTGTGGGACAAGCACGCGTGCCCAGCC |
| | | TACGTGGGACCTGAGATACTCAGCTCACGGGCCTCATACTCGGGCAAGGCAGCCGATGTCT |
| | | GGAGCCTGGGCGTGGCGCTCTTCACCATGCTGGCCGGCCACTACCCCTTCCAGGACTCGGA |
| | | GCCTGTCCTGCTCTTCGGCAAGATCCGCCGCGGGGCCTACGCCTTGCCTGCAGGCCTCTCGG |
| | | CCCCTGCCCGCTGTCTGGTTCGCTGCCTCCTTCGTCGGGAGCCAGCTGAACGGCTCACAGCC |
| | | ACAGGCATCCTCCTGCACCCCTGGCTGCGACAGGACCCGATGCCCTTAGCCCCAACCCGAT |
| | | CCCATCTCTGGGAGGCTGCCCAGGTGGTCCCTGATGGACTGGGGCTGGACGAAGCCAGGGA |
| | | AGAGGAGGGAGACAGAGAAGTGGTTCTGTATGGCTACCCAACTTTCTTGTACAAAGTTGGC |
| | | ATTATAAGAAAGCATTGCTTATCAATTTGTTGCAACGAAC |
| 15 | Homo | GTTCGTTGCAACAAATTGATGAGCAATGCTTTTTTATAATGCCAACTTTGTACAAAAAAGTT |
| | sapiens clone | GGCATGCCTTTAAACCGCACTTTGTCCATGTCCTCACTGCCAGGACTGGAGGACTGGGAGG |
| | ccsbBroadEn_15439 | ATGAATTCGACCTGGAGAACGCAGTGCTCTTCGAAGTGGCCTGGGAGGTGGCTAACAAGGT |
| | GYS1 gene. | GGGTGGCATCTACACGGTGCTGCAGACGAAGGCGAAGGTGACAGGGGACGAATGGGGCGA |
| | GenBank: | CAACTACTTCCTGGTGGGGCCGTACACGGAGCAGGGCGTGAGGACCCAGGTGGAACTGCTG |
| | KJ905769.1 | GAGGCCCCCACCCCGGCCCTGAAGAGGACACTGGATTCCATGAACAGCAAGGGCTGCAAGT |
| | | TCCTGGCACAGAGTGAGGAGAAGCCACATGTGGTTGCTCACTTCCATGAGTGGTTGGCAGG |
| | | CGTTGGACTCTGCCTGTGTCGTGCCCGGCGACTGCCTGTAGCAACCATCTTCACCACCCATG |
| | | CCACGCTGCTGGGGCGCTACCTGTGTGCCGGTGCCGTGGACTTCTACAACAACCTGGAGAA |
| | | CTTCAACGTGGACAAGGAAGCAGGGGAGAGGCAGATCTACCACCGATACTGCATGGAAAG |
| | | GGCGGCAGCCCACTGCGCTCACGTCTTCACTACTGTGTCCCAGATCACCGCCATCGAGGCAC |
| | | AGCACTTGCTCAAGAGGAAACCAGATATTGTGACCCCCAATGGGCTGAATGTGAAGAAGTT |
| | | TTCTGCCATGCATGAGTTCCAGAACCTCCATGCTCAGAGCAAGGCTCGAATCCAGGAGTTTG |
| | | TGCGGGGCCATTTTTATGGGCATCTGGACTTCAACTTGGACAAGACCTTATACTTCTTTATC |
| | | GCCGGCCGCTATGAGTTCTCCAACAAGGGTGCTGACGTCTTCCTGGAGGCATTGGCTCGGCT |
| | | CAACTATCTGCTCAGAGTGAACGGCAGCGAGCAGACAGTGGTTGCCTTCTTCATCATGCCA |
| | | GCGCGGACCAACAATTTCAACGTGGAAACCCTCAAAGGCCAAGCTGTGCGCAAACAGCTTT |
| | | GGGACACGGCCAACACGGTGAAGGAAAAGTTCGGGAGGAAGCTTTATGAATCCTTACTGGT |
| | | TGGGAGCCTTCCCGACATGAACAAGATGCTGGATAAGGAAGACTTCACTATGATGAAGAGA |
| | | GCCATCTTTGCAACGCAGCGGCAGTCTTTCCCCCCTGTGTGCACCCACAATATGCTGGATGA |
| | | CTCCTCAGACCCCATCCTGACCACCATCCGCCGAATCGGCCTCTTCAATAGCAGTGCCGACA |
| | | GGGTGAAGGTGATTTTCCACCCGGAGTTCCTCTCCTCCACAAGCCCCCTGCTCCCTGTGGAC |
| | | TATGAGGAGTTTGTCCGTGGCTGTCACCTTGGAGTCTTCCCCTCCTACTATGAGCCTTGGGG |
| | | CTACACACCGGCTGAGTGCACGGTTATGGGAATCCCCAGTATCTCCACCAATCTCTCCGGCT |
| | | TCGGCTGCTTCATGGAGGAACACATCGCAGACCCCTCAGCTTACGGTATCTACATTCTTGAC |
| | | CGGCGGTTCCGCAGCCTGGATGATTCCTGCTCGCAGCTCACCTCCTTCCTCTACAGTTTCTGT |
| | | CAGCAGAGCCGGCGGCAGCGTATCATCCAGCGGAACCGCACGGAGCGCCTCTCCGACCTTC |
| | | TGGACTGGAAATACCTAGGCCGGTACTATATGTCTGCGCGCCACATGGCGCTGTCCAAGGC |
| | | CTTTCCAGAGCACTTCACCTACGAGCCCAACGAGGCGGATGCGGCCCAGGGGTACCGCTAC |
| | | CCACGGCCAGCCTCGGTGCCACCGTCGCCCTCGCTGTCACGACACTCCAGCCCGCACCAGA |
| | | GTGAGGACGAGGAGGATCCCCGGAACGGGCCGCTGGAGGAAGACGGCGAGCGCTACGATG |
| | | AGGACGAGGAGGCCGCCAAGGACCGGCGCAACATCCGTGCACCAGAGTGGCCGCGCCGAG |
| | | CGTCCTGCACCTCCTCCACCAGCGGCAGCAAGCGCAACTCTGTGGACACGGCCACCTCCAG |
| | | CTCACTCAGCACCCCGAGCGAGCCCCTCAGCCCCACCAGCTCCCTGGGCGAGGAGCGTAAC |
| | | TNNCCAACTTTCTTGTACAAAGTTGGCATTATAAGAAAGCATTGCTTATCAATTTGTTGCAA |
| | | CGAAC |
| 16 | Homo | GTTCGTTGCAACAAATTGATGAGCAATGCTTTTTTATAATGCCAACTTTGTACAAAAAAGTT |
| | sapiens clone | GGCATGGCCGCATTGTACCGCCCTGGCCTGCGGCTTAACTGGCATGGGCTGAGCCCCTTGGG |
| | ccsbBroadEn_06695 | CTGGCCATCATGCCGTAGCATCCAGACCCTGCGAGTGCTTAGTGGAGATCTGGGCCAGCTTC |
| | PCK2 gene. | CCACTGGCATTCGAGATTTTGTAGAGCACAGTGCCCGCCTGTGCCAACCAGAGGGCATCCA |
| | GenBank: | CATCTGTGATGGAACTGAGGCTGAGAATACTGCCACACTGACCCTGCTGGAGCAGCAGGGC |
| | KJ897301.1 | CTCATCCGAAAGCTCCCCAAGTACAATAACTGCTGGCTGGCCCGCACAGACCCCAAGGATG |
| | | TGGCACGAGTAGAGAGCAAGACGGTGATTGTAACTCCTTCTCAGCGGGACACGGTACCACT |
| | | CCCGCCTGGTGGGGCCCGTGGGCAGCTGGGCAACTGGATGTCCCCAGCTGATTTCCAGCGA |
| | | GCTGTGGATGAGAGGTTTCCAGGCTGCATGCAGGGCCGCACCATGTATGTGCTTCCATTCAG |
| | | CATGGGTCCTGTGGGCTCCCCGCTGTCCCGCATCGGGGTGCAGCTCACTGACTCAGCCTATG |
| | | TGGTGGCAAGCATGCGTATTATGACCCGACTGGGGACACCTGTGCTTCAGGCCCTGGGAGA |
| | | TGGTGACTTTGTCAAGTGTCTGCACTCCGTGGGCCAGCCCCTGACAGGACAAGGGGAGCCA |
| | | GTGAGCCAGTGGCCGTGCAACCCAGAGAAAACCCTGATTGGCCACGTGCCCGACCAGCGGG |
| | | AGATCATCTCCTTCGGCAGCGGCTATGGTGGCAACTCCCTGCTGGGCAAGAAGTGCTTTGCC |
| | | CTACGCATCGCCTCTCGGCTGGCCCGGGATGAGGGCTGGCTGGCAGAGCACATGCTGATCC |
| | | TGGGCATCACCAGCCCTGCAGGGAAGAAGCGCTATGTGGCAGCCGCCTTCCCTAGTGCCTG |
| | | TGGCAAGACCAACCTGGCTATGATGCGGCCTGCACTGCCAGGCTGGAAAGTGGAGTGTGTG |
| | | GGGGATGATATTGCTTGGATGAGGTTTGACAGTGAAGGTCGACTCCGGGCCATCAACCCTG |
| | | AGAACGGCTTCTTTGGGGTTGCCCCTGGTACCTCTGCCACCACCAATCCCAACGCCATGGCT |
| | | ACAATCCAGAGTAACACTATTTTTACCAATGTGGCTGAGACCAGTGATGGTGGCGTGTACTG |
| | | GGAGGGCATTGACCAGCCTCTTCCACCTGGTGTTACTGTGACCTCCTGGCTGGGCAAACCCT |
| | | GGAAACCTGGTGACAAGGAGCCCTGTGCACATCCCAACTCTCGATTTTGTGCCCCGGCTCGC |
| | | CAGTGCCCCATCATGGACCCAGCCTGGGAGGCCCCAGAGGGTGTCCCCATTGACGCCATCA |
| | | TCTTTGGTGGCCGCAGACCCAAAGGGGTACCCCTGGTATACGAGGCCTTCAACTGGCGTCAT |
| | | GGGGTGTTTGTGGGCAGCGCCATGCGCTCTGAGTCCACTGCTGCAGCAGAACACAAAGGGA |
| | | AGATCATCATGCACGACCCATTTGCCATGCGGCCCTTTTTTGGCTACAACTTCGGGCACTAC |
| | | CTGGAACACTGGCTGAGCATGGAAGGGCGCAAGGGGGCCCAGCTGCCCCGTATCTTCCATG |
| | | TCAACTGGTTCCGGCGTGACGAGGCAGGGCACTTCCTGTGGCCAGGCTTTGGGGAGAATGC |
| | | TCGGGTGCTAGACTGGATCTGCCGGCGGTTAGAGGGGGAGGACAGTGCCCGAGAGACACCC |
| | | ATTGGGCTGGTGCCAAAGGAAGGAGCCTTGGATCTCAGCGGCCTCAGAGCTATAGACACCA |
| | | CTCAGCTGTTCTCCCTCCCCAAGGACTTCTGGGAACAGGAGGTTCGTGACATTCGGAGCTAC |
| | | CTGACAGAGCAGGTCAACCAGGATCTGCCCAAAGAGGTGTTGGCTGAGCTTGAGGCCCTGG |
| | | AGAGACGTGTGCACAAAATGTGCCCAACTTTCTTGTACAAAGTTGGCATTATAAGAAAGCA |
| | | TTGCTTATCAATTTGTTGCAACGAAC |
| 17 | Homo | GTTCGTTGCAACAAATTGATGAGCAATGCTTTTTTATAATGCCAACTTTGTACAAAAAAGTT |
| | sapiens clone | GGCATGACCCTGACTGAAAGGCTGCGTGAGAAGATATCTCGGGCCTTCTACAACCATGGGC |
| | ccsbBroadEn_11654 | TCCTCTGTGCATCCTATCCCATCCCCATCATCCTCATCGGCTACTTCACCCTAGTGCCCGCCA |
| | SCAP gene. | TCCAGGAGTTCTGTCTCTTTGCTGTCGTGGGGCTGGTGTCTGACTTCTTCCTTCAGATGCTGT |
| | GenBank: | TTTTCACCACTGTCCTGTCCATTGACATTCGCCGGATGGAGCTAGCAGACCTGAACAAGCGA |
| | KJ902260.1 | CTGCCCCCTGAGGCCTGCCTGCCCTCAGCCAAGCCAGTGGGGCAGCCAACGCGCTACGAGC |
| | | GGCAGCTGGCTGTGAGGCCGTCCACACCCCACACCATCACGTTGCAGCCGTCTTCCTTCCGA |
| | | AACCTGCGGCTCCCCAAGAGGCTGCGTGTTGTCTACTTCCTGGCCCGCACCCGCCTGGCACA |
| | | GCGCCTCATCATGGCTGGCACCGTTGTCTGGATTGGCATCCTGGTATACACAGACCCAGCAG |
| | | GGCTGCGCAACTACCTCGCTGCCCAGGTGACGGAACAGAGCCCATTGGGTGAGGGAGCCCT |
| | | GGCTCCCATGCCCGTGCCTAGTGGCATGCTGCCCCCCAGCCACCCGGACCCTGCCTTCTCCA |
| | | TCTTCCCACCTGATGCCCCTAAGCTACCTGAGAACCAGACGTCGCCAGGCGAGTCACCTGA |
| | | GCGTGGAGGTCCAGCAGAGGTTGTCCATGACAGCCCAGTCCCAGAGGTAACCTGGGGGCCT |
| | | GAGGATGAGGAACTTTGGAGGAAATTGTCCTTCCGCCACTGGCCGACGCTCTTCAGCTATTA |
| | | CAACATCACACTGGCCAAGAGGTACATCAGCCTGCTGCCCGTCATCCCAGTCACGCTCCGC |
| | | CTGAACCCGAGGGAGGCTCTGGAGGGCCGGCACCCTCAGGACGGCCGCAGTGCCTGGCCCC |
| | | CACCGGGGCCCATACCTGCTGGGCACTGGGAAGCAGGACCCAAGGGCCCAGGTGGGGTGC |
| | | AGGCCCATGGAGACGTCACGCTGTACAAGGTGGCGGCGCTGGGCCTGGCCACCGGCATCGT |
| | | CTTGGTGCTGCTGCTGCTCTGCCTCTACCGCGTGCTATGCCCGCGCAACTACGGGCAGCTGG |
| | | GTGGTGGGCCCGGGCGGCGGAGGCGCGGGGAGCTGCCCTGCGACGACTACGGCTATGCGCC |
| | | ACCCGAGACGGAGATCGTGCCGCTTGTGCTGCGCGGCCACCTCATGGACATCGAGTGCCTG |
| | | GCCAGCGACGGCATGCTGCTGGTGAGCTGCTGCCTGGCAGGCCACATCTGCGTGTGGGACG |
| | | CGCAGACCGGGGATTGCCTAACGCGCATTCCGCGCCCAGGGCAGCGCCGGGACAGTGGCGT |
| | | GGGCAGCGGGCTTGAGGCTCAGGAGAGCTGGGAACGACTTTCAGATGGTGGGAAGGCTGG |
| | | TCCAGAGGAGCCTGGGGACAGCCCTCCCCTGAGACACCGCCCCCGGGGCCCTCCGCCGCCT |
| | | TCCCTCTTCGGGGACCAGCCTGACCTCACCTGCTTAATTGACACCAACTTTTCAGCGCAGCC |
| | | TCGGTCCTCACAGCCCACTCAGCCCGAGCCCCGGCACCGGGCGGTCTGTGGCCGCTCTCGG |
| | | GACTCCCCAGGCTATGACTTCAGCTGCCTGGTGCAGCGGGTGTACCAGGAGGAGGGGCTGG |
| | | CGGCCGTCTGCACACCAGCCCTGCGCCCACCCTCGCCTGGGCCGGTGCTGTCCCAGGCCCCT |
| | | GAGGACGAGGGTGGCTCCCCCGAGAAAGGCTCCCCTTCCCTCGCCTGGGCCCCCAGTGCCG |
| | | AGGGTTCCATCTGGAGCTTGGAGCTGCAGGGCAACCTCATCGTGGTGGGGCGGAGCAGCGG |
| | | CCGGCTGGAGGTGTGGGACGCCATTGAAGGGGTGCTGTGCTGCAGCAGCGAGGAGGTCTCC |
| | | TCAGGCATTACCGCTCTGGTGTTCTTGGACAAAAGGATTGTGGCTGCACGGCTCAACGGTTC |
| | | CCTTGATTTCTTCTCCTTGGAGACCCACACTGCCCTCAGCCCCCTGCAGTTTAGAGGGACCC |
| | | CAGGGCGGGGCAGTTCCCCTGCCTCTCCAGTGTACAGCAGCAGCGACACAGTGGCCTGTCA |
| | | CCTGACCCACACAGTGCCCTGTGCACACCAAAAACCCATCACAGCCCTGAAAGCCGCTGCT |
| | | GGGCGCTTGGTGACTGGGAGCCAAGACCACACACTGAGAGTGTTCCGTCTGGAGGACTCGT |
| | | GCTGCCTCTTCACCCTTCAGGGCCACTCAGGGGCCATCACGACCGTGTACATTGACCAGACC |
| | | ATGGTGCTGGCCAGTGGAGGACAAGATGGGGCCATCTGCCTGTGGGATGTACTGACTGGCA |
| | | GCCGGGTCAGCCATGTGTTTGCTCACCGTGGGGATGTCACCTCCCTTACCTGTACCACCTCC |
| | | TGTGTCATCAGCAGTGGCCTGGATGACCTCATCAGCATCTGGGACCGCAGCACAGGCATCA |
| | | AGTTCTACTCCATTCAGCAGGACCTGGGCTGTGGTGCAAGCTTGGGTGTCATCTCAGACAAC |
| | | CTGCTGGTGACTGGCGGCCAGGGCTGTGTCTCCTTTTGGGACCTAAACTACGGGGACCTGTT |
| | | ACAGACAGTCTACCTGGGGAAGAACAGTGAGGCCCAGCCTGCCCGCCAGATCCTGGTGCTG |
| | | GACAACGCTGCCATTGTCTGCAACTTTGGCAGTGAGCTCAGCCTGGTGTATGTGCCCTCTGT |
| | | GCTGGAGAAGCTGGACTGCCCAACTTTCTTGTACAAAGTTGGCATTATAAGAAAGCATTGCT |
| | | TATCAATTTGTTGCAACGAAC |
| 18 | Homo | GTTCGTTGCAACAAATTGATGAGCAATGCTTTTTTATAATGCCAACTTTGTACAAAAAAGTT |
| | sapiens clone | GGCATGGACGAGCCACCCTTCAGCGAGGCGGCTTTGGAGCAGGCGCTGGGCGAGCCGTGCG |
| | ccsbBroadEn_06995 | ATCTGGACGCGGCGCTGCTGACCGACATCGAAGACATGCTTCAGCTTATCAACAACCAAGA |
| | SREBF1 | CAGTGACTTCCCTGGCCTATTTGACCCACCCTATGCTGGGAGTGGGGCAGGGGGCACAGAC |
| | gene. | CCTGCCAGCCCCGATACCAGCTCCCCAGGCAGCTTGTCTCCACCTCCTGCCACATTGAGCTC |
| | GenBank: | CTCTCTTGAAGCCTTCCTGAGCGGGCCGCAGGCAGCGCCCTCACCCCTGTCCCCTCCCCAGC |
| | KJ897601.1 | CTGCACCCACTCCATTGAAGATGTACCCGTCCATGCCCGCTTTCTCCCCTGGGCCTGGTATC |
| | | AAGGAAGAGTCAGTGCCACTGAGCATCCTGCAGACCCCCACCCCACAGCCCCTGCCAGGGG |
| | | CCCTCCTGCCACAGAGCTTCCCAGCCCCAGCCCCACCGCAGTTCAGCTCCACCCCTGTGTTA |
| | | GGCTACCCCAGCCCTCCGGGAGGCTTCTCTACAGGAAGCCCTCCCGGGAACACCCAGCAGC |
| | | CGCTGCCTGGCCTGCCACTGGCTTCCCCGCCAGGGGTCCCGCCCGTCTCCTTGCACACCCAG |
| | | GTCCAGAGTGTGGTCCCCCAGCAGCTACTGACAGTCACAGCTGCCCCCACGGCAGCCCCTG |
| | | TAACGACCACTGTGACCTCGCAGATCCAGCAGGTCCCGGTCCTGCTGCAGCCCCACTTCATC |
| | | AAGGCAGACTCGCTGCTTCTGACAGCCATGAAGACAGACGGAGCCACTGTGAAGGCGGCA |
| | | GGTCTCAGTCCCCTGGTCTCTGGCACCACTGTGCAGACAGGGCCTTTGCCGACCCTGGTGAG |
| | | TGGCGGAACCATCTTGGCAACAGTCCCACTGGTCGTAGATGCGGAGAAGCTGCCTATCAAC |
| | | CGGCTCGCAGCTGGCAGCAAGGCCCCGGCCTCTGCCCAGAGCCGTGGAGAGAAGCGCACA |
| | | GCCCACAACGCCATTGAGAAGCGCTACCGCTCCTCCATCAATGACAAAATCATTGAGCTCA |
| | | AGGATCTGGTGGTGGGCACTGAGGCAAAGCTGAATAAATCTGCTGTCTTGCGCAAGGCCAT |
| | | CGACTACATTCGCTTTCTGCAACACAGCAACCAGAAACTCAAGCAGGAGAACCTAAGTCTG |
| | | CGCACTGCTGTCCACAAAAGCAAATCTCTGAAGGATCTGGTGTCGGCCTGTGGCAGTGGAG |
| | | GGAACACAGACGTGCTCATGGAGGGCGTGAAGACTGAGGTGGAGGACACACTGACCCCAC |
| | | CCCCCTCGGATGCTGGCTCACCTTTCCAGAGCAGCCCCTTGTCCCTTGGCAGCAGGGGCAGT |
| | | GGCAGCGGTGGCAGTGGCAGTGACTCGGAGCCTGACAGCCCAGTCTTTGAGGACAGCAAGG |
| | | CAAAGCCAGAGCAGCGGCCGTCTCTGCACAGCCGGGGCATGCTGGACCGCTCCCGCCTGGC |
| | | CCTGTGCACGCTCGTCTTCCTCTGCCTGTCCTGCAACCCCTTGGCCTCCTTGCTGGGGGCCCG |
| | | GGGGCTTCCCAGCCCCTCAGATACCACCAGCGTCTACCATAGCCCTGGGCGCAACGTGCTG |
| | | GGCACCGAGAGCAGAGATGGCCCTGGCTGGGCCCAGTGGCTGCTGCCCCCAGTGGTCTGGC |
| | | TGCTCAATGGGCTGTTGGTGCTCGTCTCCTTGGTGCTTCTCTTTGTCTACGGTGAGCCAGTCA |
| | | CACGGCCCCACTCAGGCCCCGCCGTGTACTTCTGGAGGCATCGCAAGCAGGCTGACCTGGA |
| | | CCTGGCCCGGGGAGACTTTGCCCAGGCTGCCCAGCAGCTGTGGCTGGCCCTGCGGGCACTG |
| | | GGCCGGCCCCTGCCCACCTCCCACCTGGACCTGGCTTGTAGCCTCCTCTGGAACCTCATTCG |
| | | TCACCTGCTGCAGCGTCTCTGGGTGGGCCGCTGGCTGGCAGGCCGGGCAGGGGGCCTGCAG |
| | | CAGGACTGTGCTCTGCGAGTGGATGCTAGCGCCAGCGCCCGAGACGCAGCCCTGGTCTACC |
| | | ATAAGCTGCACCAGCTGCACACCATGGGGAAGCACACAGGCGGGCACCTCACTGCCACCAA |
| | | CCTGGCGCTGAGTGCCCTGAACCTGGCAGAGTGTGCAGGGGATGCCGTGTCTGTGGCGACG |
| | | CTGGCCGAGATCTATGTGGCGGCTGCATTGAGAGTGAAGACCAGTCTCCCACGGGCCTTGC |
| | | ATTTTCTGACACGCTTCTTCCTGAGCAGTGCCCGCCAGGCCTGCCTGGCACAGAGTGGCTCA |
| | | GTGCCTCCTGCCATGCAGTGGCTCTGCCACCCCGTGGGCCACCGTTTCTTCGTGGATGGGGA |
| | | CTGGTCCGTGCTCAGTACCCCATGGGAGAGCCTGTACAGCTTGGCCGGGAACCCAGTGGAC |
| | | CCCCTGGCCCAGGTGACTCAGCTATTCCGGGAACATCTCTTAGAGCGAGCACTGAACTGTGT |
| | | GACCCAGCCCAACCCCAGCCCTGGGTCAGCTGATGGGGACAAGGAATTCTCGGATGCCCTC |
| | | GGGTACCTGCAGCTGCTGAACAGCTGTTCTGATGCTGCGGGGGCTCCTGCCTACAGCTTCTC |
| | | CATCAGTTCCAGCATGGCCACCACCACCGGCGTAGACCCGGTGGCCAAGTGGTGGGCCTCT |
| | | CTGACAGCTGTGGTGATCCACTGGCTGCGGCGGGATGAGGAGGCGGCTGAGCGGCTGTGCC |
| | | CGCTGGTGGAGCACCTGCCCCGGGTGCTGCAGGAGTCTGAGAGACCCCTGCCCAGGGCAGC |
| | | TCTGCACTCCTTCAAGGCTGCCCGGGCCCTGCTGGGCTGTGCCAAGGCAGAGTCTGGTCCAG |
| | | CCAGCCTGACCATCTGTGAGAAGGCCAGTGGGTACCTGCAGGACAGCCTGGCTACCACACC |
| | | AGCCAGCAGCTCCATTGACAAGGCCGTGCAGCTGTTCCTGTGTGACCTGCTTCTTGTGGTGC |
| | | GCACCAGCCTGTGGCGGCAGCAGCAGCCCCCGGCCCCGGCCCCAGCAGCCCAGGGCACCA |
| | | GCAGCAGGCCCCAGGCTTCCGCCCTTGAGCTGCGTGGCTTCCAACGGGACCTGAGCAGCCT |
| | | GAGGCGGCTGGCACAGAGCTTCCGGCCCGCCATGCGGAGGGTGTTCCTACATGAGGCCACG |
| | | GCCCGGCTGATGGCGGGGGCCAGCCCCACACGGACACACCAGCTCCTCGACCGCAGTCTGA |
| | | GGCGGCGGGCAGGCCCCGGTGGCAAAGGAGGCGCGGTGGCGGAGCTGGAGCCGCGGCCCA |
| | | CGCGGCGGGAGCACGCGGAGGCCTTGCTGCTGGCCTCCTGCTACCTGCCCCCCGGCTTCCTG |
| | | TCGGCGCCCGGGCAGCGCGTGGGCATGCTGGCTGAGGCGGCGCGCACACTCGAGAAGCTTG |
| | | GCGATCGCCGGCTGCTGCACGACTGTCAGCAGATGCTCATGCGCCTGGGCGGTGGGACCAC |
| | | TGTCACTTCCAGCTACCCAACTTTCTTGTACAAAGTTGGCATTATAAGAAAGCATTGCTTAT |
| | | CAATTTGTTGCAACGAAC |
| 19 | Homo | CTAATGCCTGTGGATGAGAAAAGAGGCTTAGAGAAATTATGTTTTGCCTAATTGACAAAAC |
| | sapiens Rap | CAATTAGTGGCTGAAACAACACAGAAGGCCAGGTTTGTCCTCTCACTTCCTAACTTGAGGAC |
| | guanine | AGAGAAATATATTCTGATGTCCCTAACTGGCAGTCTGCTATGATAGTGACCACATGCCCATT |
| | nucleotide | TCTTTCTTGGGATTTCGTCCCAGCCTGACAACCTTGGAACAGAACAAGAGGACAGGAGGTG |
| | exchange | TCGTTCCAGACTCTCAGCGTTCTCATCTCTCTTCCTTCACCATGAAGCTGATGGACAAATTCC |
| | factor 1 | ACTCACCCAAAATCAAGAGAACGCCATCAAAGAAGGGAAAACCAGCTGAGGTGTCCGTAA |
| | (RAPGEF1), | AGATTCCAGAGAAGCCTGTGAACAAAGAATCCAGGTTTCCTCTCTTCCAGGAGGCAACAGA |
| | transcript. | CAGATTTCTACCAGAGGGCTACCCTCTCCCCTTGGATCTGGAGCAGCAGGCAGTAGAATTTA |
| | NCBI | TGTCCACCAGTGCTGTGGCTTCCAGGTCTCAAAGGCAGAAGAACCTGAGCTGGCTGGAGGA |
| | Reference | GAAAGAGAAGGAAGTTGTCAGTGCCCTGCGCTACTTTAAGACCATTGTGGACAAAATGGCA |
| | Sequence: | ATTGATAAGAAGGTACTGGAGATGCTTCCAGGGTCAGCCAGCAAGGTGCTGGAGGCCATCT |
| | XM_011518581.3 | TACCCCTGGTGCAGAACGATCCTCGAATTCAGCACAGCTCAGCCCTCTCTTCCTGCTATAGC |
| | | CGAGTGTACCAAAGCCTCGCCAACCTCATTCGCTGGTCTGACCAAGTGATGCTGGAAGGCG |
| | | TGAACTCAGAAGACAAGGAGATGGTGACGACTGTGAAGGGGGTCATCAAGGCTGTGCTGG |
| | | ATGGAGTGAAGGAGCTGGTCAGGCTCACCATCGAGAAGCAGGGACGTCCGTCTCCGACGAG |
| | | CCCCGTGAAGCCCAGTTCCCCTGCCAGCAAGCCTGATGGCCCAGCAGAGCTCCCCCTGACA |
| | | GACCGCGAGGTAGAGATCCTAAACAAGACGACTGGGATGTCACAGTCAACTGAGCTCCTCC |
| | | CAGATGCCACGGATGAAGAGGTCGCGCCCCCCAAGCCTCCTCTGCCTGGCATTCGGGTGGT |
| | | TGATAATAGTCCTCCACCAGCATTGCCACCCAAGAAAAGACAGTCGGCGCCGTCCCCTACC |
| | | CGAGTGGCTGTGGTGGCCCCCATGAGCCGAGCCACCAGTGGCTCCAGTTTGCCTGTTGGAAT |
| | | CAATAGGCAGGATTTTGATGTTGACTGTTACGCACAGAGGCGACTGTCAGGAGGCAGCCAC |
| | | TCATATGGTGGAGAGTCGCCCCGCCTCTCCCCCTGCAGCAGCATAGGCAAGCTCAGCAAGT |
| | | CAGACGAGCAGCTGTCCTCTCTGGACAGGGACAGTGGGCAGTGCTCCCGGAACACAAGCTG |
| | | TGAAACACTAGACCACTATGATCCCGACTATGAATTCCTCCAGCAAGACCTCTCTAACGCAG |
| | | ACCAGATACCTCAGCAGACGGCCTGGAACCTTAGCCCGTTGCCAGAGTCTTTGGGGGAGTC |
| | | TGGGTCTCCATTTCTTGGCCCTCCTTTCCAGCTGCCTCTTGGCGGCCATCCCCAGCCAGACGG |
| | | ACCTCTGGCCCCAGGGCAGCAGACAGATACGCCACCTGCTCTCCCCGAGAAGAAGCGCAGG |
| | | AGCGCAGCCTCCCAGACGGCGGACGGCTCTGGCTGCAGGGTGTCCTACGAGCGGCATCCCT |
| | | CGCAGTATGACAACATCTCTGGGGAGGACCTGCAGAGCACAGCCCCGATCCCATCCGTCCC |
| | | CTACGCGCCCTTTGCTGCTATTCTGCCCTTTCAGCATGGAGGTTCCTCAGCCCCTGTCGAATT |
| | | TGTGGGTGATTTTACTGCTCCTGAGTCAACCGGTGACCCAGAAAAACCACCTCCTCTACCAG |
| | | AGAAGAAAAACAAACACATGCTGGCCTACATGCAGTTGCTGGAGGACTACTCGGAGCCGCA |
| | | GCCCTCTATGTTCTACCAGACGCCACAGAACGAGCACATCTACCAGCAGAAGAACAAGCTC |
| | | CTCATGGAGGTATACGGCTTCAGCGACTCCTTCAGTGGGGTGGACTCCGTGCAGGAGCTGG |
| | | CCCCGCCGCCCGCCCTACCCCCCAAGCAGCGGCAGCTGGAGCCACCGGCTGGGAAAGACGG |
| | | ACATCCCAGAGATCCCTCAGCGGTCAGCGGCGTCCCTGGGAAGGACAGCAGAGACGGCAGT |
| | | GAGAGCGGAATCACTTCCGTGTACAGATGGAACATCCAGAGGCAGCAGCGACTGTCCTATG |
| | | GCCACACAGCCAGTCAGTAGCAGAGCTGAGGATCCAGTCCATAGTGACGTAGCTGCCATCG |
| | | GGGCAGGGCCCCAAAGTCACCAGATGCTCTGGAGTCGGCTCAGTCGGAGGAGGAAGTGGA |
| | | CGAGCTGTCCCTCATTGACCA |
| 20 | Homo | GTTCCCAGGACGGAAGTGGCCGAGAGAGTGTCGAAGGGAGGGCGAGGCCGGAGCCCGAGG |
| | sapiens talin | GCGACCCGAGAAGCGGCGGGGCGGCGGGCCGGCGGGCGGGGCGCAGAGCCAGGCAGCGC |
| | 1 (TLN1), | AGGTATAGCCAGGCTGGAGAAAAGAAGCTGCCACCATGGTTGCACTTTCACTGAAGATCAG |
| | mRNA. | CATTGGGAATGTGGTGAAGACGATGCAGTTTGAGCCGTCTACCATGGTGTACGACGCCTGC |
| | NCBI | CGCATCATTCGTGAGCGGATCCCAGAGGCCCCAGCTGGTCCTCCCAGCGACTTTGGGCTCTT |
| | Reference | TCTGTCAGATGATGACCCCAAAAAGGGTATATGGCTGGAGGCTGGGAAAGCTTTGGACTAC |
| | Sequence: | TACATGCTCCGAAATGGGGACACTATGGAGTACAGGAAGAAACAGAGACCCCTGAAGATC |
| | NM_006289.4 | CGTATGCTGGATGGAACTGTGAAGACGATCATGGTGGATGACTCTAAGACTGTCACTGACA |
| | | TGCTCATGACCATCTGTGCCCGCATTGGCATCACCAATCATGATGAATATTCATTGGTTCGA |
| | | GAGCTGATGGAAGAGAAAAAGGAGGAAATAACAGGGACCTTAAGAAAGGACAAGACATTG |
| | | CTGCGAGATGAAAAGAAGATGGAGAAACTAAAGCAGAAATTGCACACAGATGATGAGTTG |
| | | AACTGGCTGGACCATGGTCGGACACTGAGGGAGCAGGGTGTAGAGGAGCACGAGACGCTG |
| | | CTGCTGCGGAGGAAGTTCTTTTACTCAGACCAGAATGTGGATTCCCGGGACCCTGTACAGCT |
| | | GAACCTCCTGTATGTGCAGGCACGAGATGACATCCTGAATGGCTCCCACCCTGTCTCCTTTG |
| | | ACAAGGCCTGTGAGTTTGCTGGCTTCCAATGCCAGATCCAGTTTGGGCCCCACAATGAGCA |
| | | GAAGCACAAGGCTGGCTTCCTTGACCTGAAGGACTTCCTGCCCAAGGAGTATGTGAAGCAG |
| | | AAGGGAGAGCGTAAGATCTTCCAGGCACACAAGAATTGTGGGCAGATGAGTGAGATTGAG |
| | | GCCAAGGTCCGCTACGTGAAGCTAGCCCGTTCTCTCAAGACTTACGGTGTCTCCTTCTTCCT |
| | | GGTGAAGGAAAAAATGAAAGGGAAGAACAAGCTAGTGCCCAGGCTTCTGGGCATCACCAA |
| | | GGAGTGTGTGATGCGAGTGGATGAGAAGACCAAGGAAGTGATCCAGGAGTGGAACCTCAC |
| | | CAACATCAAACGCTGGGCTGCGTCTCCCAAAAGCTTCACCCTGGATTTTGGAGATTACCAAG |
| | | ATGGCTATTACTCAGTACAGACAACTGAAGGGGAGCAGATTGCACAGCTCATTGCCGGCTA |
| | | CATCGATATCATCCTGAAGAAGAAAAAAAGCAAGGATCACTTTGGGCTGGAAGGAGATGA |
| | | GGAGTCTACTATGCTGGAGGACTCAGTGTCCCCCAAAAAGTCAACAGTCCTGCAGCAGCAA |
| | | TACAACCGGGTGGGGAAAGTGGAGCATGGCTCTGTGGCCCTGCCTGCCATCATGCGCTCTG |
| | | GAGCCTCTGGTCCTGAGAATTTCCAGGTGGGCAGCATGCCCCCTGCCCAGCAGCAGATTAC |
| | | CAGCGGCCAGATGCACCGAGGACACATGCCTCCTCTGACTTCAGCCCAGCAGGCACTCACT |
| | | GGAACCATTAACTCCAGCATGCAGGCCGTGCAGGCTGCCCAGGCCACCCTGGATGACTTTG |
| | | ACACTCTGCCGCCTCTTGGCCAGGATGCTGCCTCTAAGGCCTGGCGTAAAAACAAGATGGA |
| | | TGAATCAAAGCATGAGATCCACTCTCAGGTAGATGCCATCACAGCTGGTACTGCGTCTGTG |
| | | GTGAACCTGACAGCAGGGGACCCTGCTGAGACAGACTATACCGCAGTGGGCTGTGCAGTCA |
| | | CCACAATCTCCTCCAACCTGACGGAGATGTCCCGTGGGGTGAAGCTGCTGGCTGCCTTGCTG |
| | | GAGGACGAAGGCGGCAGTGGTCGGCCCCTGTTGCAGGCAGCAAAGGGCCTTGCGGGAGCA |
| | | GTGTCAGAACTGCTGCGCAGTGCCCAACCAGCCAGTGCTGAGCCCCGTCAGAACCTGCTGC |
| | | AAGCAGCTGGGAACGTGGGCCAGGCCAGTGGGGAGCTGTTGCAACAAATTGGGGAAAGTG |
| | | ATACTGACCCCCACTTCCAGGATGCGCTAATGCAGCTCGCCAAAGCTGTGGCAAGTGCTGC |
| | | AGCTGCCCTGGTCCTCAAGGCCAAGAGTGTGGCCCAGCGGACAGAGGACTCGGGACTTCAG |
| | | ACCCAAGTTATTGCTGCAGCAACACAGTGTGCCCTATCCACTTCCCAACTAGTGGCCTGTAC |
| | | TAAGGTGGTGGCACCTACAATCAGCTCACCTGTCTGCCAAGAGCAACTGGTGGAGGCTGGA |
| | | CGACTGGTAGCCAAAGCCGTGGAGGGCTGTGTGTCTGCCTCCCAGGCAGCTACAGAGGATG |
| | | GGCAACTGTTGCGAGGGGTAGGAGCAGCAGCCACAGCTGTCACCCAGGCCCTAAATGAGCT |
| | | GCTGCAGCATGTGAAAGCCCATGCCACAGGGGCTGGGCCTGCTGGCCGTTATGACCAGGCT |
| | | ACTGACACCATCCTAACCGTCACTGAGAACATCTTTAGCTCCATGGGTGATGCTGGGGAGAT |
| | | GGTGCGACAGGCCCGCATCCTGGCCCAAGCCACATCTGACCTGGTCAATGCCATCAAGGCT |
| | | GATGCTGAGGGGGAAAGTGATCTGGAGAACTCCCGCAAGCTCTTAAGTGCTGCCAAGATCC |
| | | TAGCTGATGCCACAGCCAAGATGGTAGAGGCTGCCAAGGGAGCAGCTGCCCACCCTGACAG |
| | | TGAGGAGCAGCAGCAGCGGCTGCGGGAGGCAGCTGAGGGGCTGCGCATGGCCACCAATGC |
| | | AGCTGCGCAGAATGCCATCAAGAAAAAGCTGGTGCAGCGCCTGGAGCATGCAGCCAAGCA |
| | | GGCTGCAGCCTCAGCCACACAGACCATCGCTGCAGCTCAGCACGCAGCCTCTACCCCCAAG |
| | | GCCTCTGCCGGCCCCCAGCCCCTGCTGGTGCAGAGCTGCAAGGCAGTGGCAGAGCAGATTC |
| | | CACTGCTGGTGCAGGGCGTCCGAGGAAGCCAAGCCCAGCCTGACAGCCCCAGCGCTCAGCT |
| | | TGCCCTCATTGCTGCCAGCCAGAGCTTCCTGCAGCCAGGTGGGAAGATGGTGGCAGCTGCA |
| | | AAGGCCTCAGTGCCAACGATTCAGGACCAGGCTTCAGCCATGCAGCTGAGTCAGTGTGCCA |
| | | AGAACCTGGGCACCGCGCTGGCTGAACTCCGGACGGCTGCCCAGAAGGCTCAGGAAGCATG |
| | | TGGACCTTTGGAGATGGATTCTGCACTGAGTGTGGTACAGAATCTAGAGAAAGATCTACAG |
| | | GAAGTGAAGGCAGCAGCTCGAGATGGCAAGCTTAAACCCTTACCTGGGGAGACAATGGAG |
| | | AAGTGTACCCAGGACCTGGGCAACAGCACCAAAGCCGTGAGCTCAGCCATCGCCCAGCTAC |
| | | TGGGAGAGGTTGCCCAGGGCAATGAGAATTATGCAGGTATTGCAGCTCGGGATGTGGCAGG |
| | | TGGGCTGCGGTCACTGGCCCAGGCCGCTAGGGGAGTCGCTGCACTGACGTCAGATCCTGCA |
| | | GTGCAGGCCATTGTACTTGATACGGCCAGTGATGTGCTGGACAAGGCCAGCAGCCTCATTG |
| | | AGGAGGCGAAAAAGGCAGCTGGCCATCCAGGGGACCCTGAGAGCCAGCAGCGGCTTGCCC |
| | | AGGTGGCTAAAGCAGTGACCCAGGCTCTGAACCGCTGTGTCAGCTGCCTACCTGGCCAGCG |
| | | CGATGTGGATAATGCCCTGAGGGCAGTTGGAGATGCCAGCAAGCGACTCCTGAGTGACTCG |
| | | CTTCCTCCTAGCACTGGGACATTTCAAGAAGCTCAGAGCCGGTTGAATGAAGCTGCTGCTGG |
| | | GCTGAATCAGGCAGCCACAGAACTGGTGCAGGCCTCTCGGGGAACCCCTCAGGACCTGGCT |
| | | CGAGCCTCAGGCCGATTTGGACAGGACTTCAGCACCTTCCTGGAAGCTGGTGTGGAGATGG |
| | | CAGGCCAGGCTCCGAGCCAGGAGGACCGAGCCCAAGTTGTGTCCAACTTGAAGGGCATCTC |
| | | CATGTCTTCAAGCAAACTTCTTCTGGCTGCCAAGGCCCTGTCCACGGACCCTGCTGCCCCTA |
| | | ACCTCAAGAGTCAGCTGGCTGCAGCTGCCAGGGCAGTAACTGACAGCATCAATCAGCTCAT |
| | | CACTATGTGCACCCAGCAGGCACCCGGCCAGAAGGAGTGTGATAACGCCCTGCGGGAATTG |
| | | GAGACGGTCCGGGAACTCCTGGAGAACCCAGTCCAGCCCATCAATGACATGTCCTACTTTG |
| | | GTTGCCTGGACAGTGTAATGGAGAACTCAAAGGTGCTGGGCGAGGCCATGACTGGCATCTC |
| | | CCAAAATGCCAAGAACGGAAACCTGCCAGAGTTTGGAGATGCCATTTCCACAGCCTCAAAG |
| | | GCACTTTGTGGCTTCACCGAGGCAGCTGCACAGGCTGCATATCTGGTTGGTGTCTCTGACCC |
| | | CAATAGCCAAGCTGGACAGCAAGGGCTAGTGGAGCCCACACAGTTTGCCCGTGCAAACCAG |
| | | GCAATTCAGATGGCCTGCCAGAGTTTGGGAGAGCCTGGCTGTACCCAGGCCCAGGTGCTCT |
| | | CTGCAGCCACCATTGTGGCTAAACACACCTCTGCACTGTGTAACAGCTGTCGCCTGGCTTCT |
| | | GCCCGTACCACCAATCCTACTGCCAAGCGCCAGTTTGTACAGTCAGCCAAGGAGGTGGCCA |
| | | ACAGCACAGCTAATCTTGTCAAGACCATCAAGGCGCTAGATGGGGCCTTCACAGAGGAGAA |
| | | CCGTGCCCAGTGCCGAGCAGCAACAGCCCCTCTGCTGGAGGCTGTGGACAATCTGAGTGCC |
| | | TTTGCGTCCAACCCTGAGTTCTCCAGCATTCCTGCCCAGATCAGCCCTGAGGGTCGGGCTGC |
| | | CATGGAGCCCATTGTGATCTCTGCCAAGACAATGTTAGAGAGTGCCGGGGGACTCATCCAG |
| | | ACAGCCCGGGCCCTCGCAGTCAATCCCCGGGACCCCCCGAGCTGGTCGGTGCTGGCCGGCC |
| | | ACTCCCGTACTGTCTCAGACTCCATCAAGAAGCTAATTACAAGCATGAGGGACAAGGCTCC |
| | | AGGGCAGCTGGAGTGTGAAACGGCCATTGCAGCTCTGAACAGTTGTCTACGGGACCTAGAC |
| | | CAGGCTTCCCTCGCTGCAGTCAGCCAGCAGCTTGCTCCCCGTGAGGGAATCTCTCAAGAGGC |
| | | CTTGCACACTCAGATGCTCACTGCAGTCCAAGAGATCTCCCATCTCATTGAGCCGCTGGCCA |
| | | ATGCTGCCCGGGCTGAAGCCTCCCAGCTGGGACACAAGGTGTCCCAGATGGCGCAGTACTT |
| | | TGAGCCGCTCACCCTGGCTGCAGTGGGTGCTGCCTCCAAGACCCTGAGCCACCCGCAGCAG |
| | | ATGGCACTCCTGGACCAGACTAAAACATTGGCAGAGTCTGCCCTGCAGTTGCTATACACTGC |
| | | CAAGGAGGCTGGTGGTAACCCAAAGCAAGCAGCTCACACCCAGGAAGCCCTGGAGGAGGC |
| | | TGTGCAGATGATGACCGAGGCCGTAGAGGACCTGACAACAACCCTCAACGAGGCAGCCAGT |
| | | GCTGCTGGGGTCGTGGGTGGCATGGTGGACTCCATCACCCAGGCCATCAACCAGCTAGATG |
| | | AAGGACCAATGGGTGAACCAGAAGGTTCCTTCGTGGATTACCAAACAACTATGGTGCGGAC |
| | | AGCCAAGGCCATTGCAGTGACCGTTCAGGAGATGGTTACCAAGTCAAACACCAGCCCAGAG |
| | | GAGCTGGGCCCTCTTGCTAACCAGCTGACCAGTGACTATGGCCGTCTGGCCTCGGAGGCCA |
| | | AGCCTGCAGCGGTGGCTGCTGAAAATGAAGAGATAGGTTCCCATATCAAACACCGGGTACA |
| | | GGAGCTGGGCCATGGCTGTGCCGCTCTGGTCACCAAGGCAGGCGCCCTGCAGTGCAGCCCC |
| | | AGTGATGCCTACACCAAGAAGGAGCTCATAGAGTGTGCCCGGAGAGTCTCTGAGAAGGTCT |
| | | CCCACGTCCTGGCTGCGCTCCAGGCTGGGAATCGTGGCACCCAGGCCTGCATCACAGCAGC |
| | | CAGCGCTGTGTCTGGTATCATTGCTGACCTCGACACCACCATCATGTTCGCCACTGCTGGCA |
| | | CGCTCAATCGTGAGGGTACTGAAACTTTCGCTGACCACCGGGAGGGCATCCTGAAGACTGC |
| | | GAAGGTGCTGGTGGAGGACACCAAGGTCCTGGTGCAAAACGCAGCTGGGAGCCAGGAGAA |
| | | GTTGGCGCAGGCTGCCCAGTCCTCCGTGGCGACCATCACCCGCCTCGCTGATGTGGTCAAGC |
| | | TGGGTGCAGCCAGCCTGGGAGCTGAGGACCCTGAGACCCAGGTGGTACTAATCAACGCAGT |
| | | GAAAGATGTAGCCAAAGCCCTGGGAGACCTCATCAGTGCAACGAAGGCTGCAGCTGGCAA |
| | | AGTTGGAGATGACCCTGCTGTGTGGCAGCTAAAGAACTCTGCCAAGGTGATGGTGACCAAT |
| | | GTGACATCATTGCTTAAGACAGTAAAAGCCGTGGAAGATGAGGCCACCAAAGGCACTCGGG |
| | | CCCTGGAGGCAACCACAGAACACATACGGCAGGAGCTGGCGGTTTTCTGTTCCCCAGAGCC |
| | | ACCTGCCAAGACCTCTACCCCAGAAGACTTCATCCGAATGACCAAGGGTATCACCATGGCA |
| | | ACCGCCAAGGCCGTTGCTGCTGGCAATTCCTGTCGCCAGGAAGATGTCATTGCCACAGCCA |
| | | ATCTGAGCCGCCGTGCTATTGCAGATATGCTTCGGGCTTGCAAGGAAGCAGCTTACCACCCA |
| | | GAAGTGGCCCCTGATGTGCGGCTTCGAGCCCTGCACTATGGCCGGGAGTGTGCCAATGGCT |
| | | ACCTGGAACTGCTGGACCATGTACTGCTGACCCTGCAGAAGCCAAGCCCAGAACTGAAGCA |
| | | GCAGTTGACAGGACATTCAAAGCGTGTGGCTGGTTCCGTCACTGAGCTCATCCAGGCTGCTG |
| | | AAGCCATGAAGGGAACAGAATGGGTAGACCCAGAGGACCCCACAGTCATTGCTGAGAATG |
| | | AGCTCCTGGGAGCTGCAGCCGCCATTGAGGCTGCAGCCAAAAAGCTAGAGCAGCTGAAGCC |
| | | CCGGGCCAAACCCAAGGAGGCAGATGAGTCCTTGAACTTTGAGGAGCAGATACTAGAAGCT |
| | | GCCAAGTCCATTGCAGCAGCCACCAGTGCACTGGTAAAGGCTGCGTCGGCTGCCCAGAGAG |
| | | AACTAGTGGCCCAAGGGAAGGTGGGTGCCATTCCAGCCAATGCACTGGACGATGGGCAGTG |
| | | GTCCCAGGGCCTCATTTCTGCTGCCCGGATGGTGGCTGCGGCCACCAACAATCTGTGTGAGG |
| | | CAGCCAATGCAGCTGTACAAGGCCATGCCAGCCAGGAGAAGCTCATCTCATCAGCCAAGCA |
| | | GGTAGCTGCCTCCACAGCCCAGCTCCTTGTGGCCTGCAAGGTCAAGGCTGACCAGGACTCG |
| | | GAGGCAATGAAACGACTTCAGGCTGCTGGCAACGCAGTGAAGCGAGCCTCAGATAATCTGG |
| | | TGAAAGCAGCACAGAAGGCTGCAGCCTTTGAAGAGCAGGAGAATGAGACAGTGGTGGTGA |
| | | AAGAGAAGATGGTTGGCGGCATTGCCCAGATCATCGCAGCACAGGAAGAAATGCTTCGGA |
| | | AGGAACGAGAGCTGGAAGAGGCGCGGAAGAAACTGGCCCAGATCCGGCAGCAGCAGTACA |
| | | AGTTTCTGCCTTCAGAGCTTCGAGATGAGCACTAAAGAAGCCTCTTCTATTTAATGCAGACC |
| | | CGGCCCAGAGACTGTGCGTGCCACTACCAAAGCCTTCTGGGCTGTCGGGGCCCAACCTGCC |
| | | CAACCCCAGCACTCCCCAAAGTGCCTGCCAAACCCCAGGGCCTGGCCCCGCCCAGTCCCGC |
| | | AGTACATCCCCTGTCCCCTCCCCAACCCCAAGTGCCTTCATGCCCTAGGGCCCCCCAAGTGC |
| | | CTGCCCCTCCCCAGAGTATTAACGCTCCAAGAGTATTATTAACGCTGCTGTACCTCGATCTG |
| | | AATCTGCCGGGGCCCCAGCCCACTCCACCCTGCCAGCAGCTTCCGGCCAGTCCCCACAGCCT |
| | | CATCAGCTCTCTTCACCGTTTTTTGATACTATCTTCCCCCACCCCCAGCTACCCATAGGGGCT |
| | | GCAGAGTTATAAGCCCCAAACAGGTCATGCTCCAATAAAAATGATTCTACCTACAACCTCT |
| | | GCCTGGCTTCAAGGGAGATACAAGTTTTCTCCCAGGGCAGTAGGAGAGACAGTGGGGTTGA |
| | | ATTCTGTCACCCAGCTTGATCCAGCTTGAAATGGGAGAGGGGAAGACTGGCAGTCTGCACC |
| | | TGAGGTCCCTTCCTCCTTGCACCCGGACCCTAAAGTTATCCAATGGGGAAGTTGTGCCTGAG |
| | | AAGTCATACTCCTGTTATCCTCACCTCCCTGGCCAGCACCTAAGCCTCACAAAGTGTCGTTC |
| | | TGCTGCTGCTGGGACAGCAGCTACCACAGTTCCTTGTGGCTACCAGGACATTCTGTTCTTCA |
| | | TAGCTACTCAGGGTATGTCAACAAATCCAGTCCTTGACCAAATTCAAATTTTCAAAAGAAGC |
| | | TTTGCTAAAAATAA |
| 21 | Homo | CGGCGCGGGCGGGCCGGCCGGGCTGTGCACCTGCGCCTCGGCGGGCCGCCTGGGGCACCGT |
| | sapiens par-6 | CCCCGGCCC |
| | family cell | GCCCGGCCCCGCCATGGCCCGGCCGCAGAGGACTCCGGCGCGCAGTCCCGATAGCATCGTC |
| | polarity | GAGGTGAAG |
| | regulator | AGCAAATTTGACGCCGAGTTCCGACGCTTCGCGCTGCCTCGCGCTTCGGTGAGCGGCTTCCA |
| | alpha | GGAGTTCT |
| | (PARD6A), | CGCGGTTGCTGCGGGCGGTGCACCAGATCCCGGGCCTGGACGTGCTACTTGGCTATACGGA |
| | transcript. | TGCTCATGG |
| | NCBI | CGACCTGCTGCCCCTCACCAACGACGACAGCCTGCACCGGGCCCTGGCCAGCGGGCCCCCG |
| | Reference | CCACTGCGCCTACTGGTGCAGAAGCGGGGTGAGGAGGGGTACAGTGGGCAGCCTCTGTGGG |
| | Sequence: | AAGCTGACTCCAGCGGCCTGGCTTTTGCCTCCAACTCTCTGCAGCGGCGCAAGAAAGGGCT |
| | XM_011523095.2 | CTTGCTGCGGCCAGTGGCACCCCTGCGCACCCGGCCACCCTTGCTAATCAGCCTGCCCCAAG |
| | | ATTTCCGCCAGGTTTCCTCAGTCATAGACGTGGACCTACTGCCTGAGACCCACCGACGGGTG |
| | | CGGCTGCACAAGCATGGTTCAGACCGCCCCCTGGGCTTCTACATCCGAGATGGCATGAGCG |
| | | TGCGTGTGGCTCCCCAGGGCCTGGAGCGGGTTCCAGGAATCTTCATCTCCCGCCTGGTACGT |
| | | GGGGGTCTGGCTGAGAGTACAGGGCTGCTGGCGGTCAGTGATGAGATCCTCGAGGTCAAT |
| | | GGCATTGAAGTAGCCGGGAAGACCTTGGACCAAGTGACGGACATGATGGTTGCCAACAGCC |
| | | ATAACCTCATTGTCACTGTCAAGCCCGCCAACCAGCGCAATAACGTGGTGCGAGGGGCATC |
| | | TGGGCGTTTGACAGGTCCTCCCTCTGCAGGGCCTGGGCCTGCTGAGCCTGATAGTGACGATG |
| | | ACAGCAGTGACCTGGTCATTGAGAACCGCCAGCCTCCCAGTTCCAATGGGCTGTCTCAGGG |
| | | GCCCCCGTGCTGGGACCTGCACCCTGGCTGCCGACATCCTGGTACCCGCAGCTCTCTGCCCT |
| | | CCCTGGATGACCAGGAGCAGGCCAGTTCTGGCTGGGGGAGTCGCATTCGAGGAGATGGTAG |
| | | TGGCTTCAGCCTCTGACAGTCAGGATGAAGCCCCATGCCACTCCACACTGCTGGGACATGG |
| | | CAGGGACTTCACAGTGGGGGTTTTTAGCTGGCTCACAGGGCTCCCTCAGCCTGGGGAACAT |
| | | TAAAGGTTTTCTACAAATACA |
| 22 | Homo | TGGCTTGATTGGCGTGCTGAGACGCACCTGGCGCAACCCTCCCTTCTGAATCGAAGTTCAAG |
| | sapiens | TCCCGCGGACACTGCAACCATGAAGGAGAGACGGGCCCCCCAGCCAGTCGTGGCCAGATGT |
| | mRNA for | AAGCTCGTTCTGGTCGGGGACGTGCAGTGTGGGAAGACCGCGATGTTGCAAGTGTTAGCGA |
| | Rnd1, | AGGATTGCTATCCAGAGACCTATGTGCCCACCGTGTTCGAAAATTACACAGCCTGTTTGGAG |
| | complete | ACAGAGGAACAGAGGGTGGAGCTTAGTCTCTGGGATACCTCAGGATCTCCCTACTACGATA |
| | cds. | ATGTCCGTCCACTCTGCTACAGCGACTCGGATGCAGTATTACTATGTTTTGACATCAGCCGT |
| | GenBank: | CCAGAGACAGTGGACAGCGCACTCAAGAAGTGGAGGACAGAAATCCTAGATTATTGTCCCA |
| | AB040147.1 | GCACCCGCGTTTTGCTCATTGGCTGCAAGACAGACCTGCGAACAGACCTGAGTACTCTGATG |
| | | GAGCTGTCCCACCAGAAGCAGGCGCCCATCTCCTATGAGCAGGGTTGTGCAATAGCAAAGC |
| | | AGCTGGGTGCAGAAATCTACCTGGAAGGCTCAGCTTTCACCTCAGAAAAGAGCATCCACAG |
| | | CATCTTTCGGACGGCATCCATGCTGTGTCTGAACAAGCCTAGCCCACTGCCCCAGAAGAGCC |
| | | CTGTCCGAAGCCTCTCCAAACGGCTGCTCCACCTCCCCAGTCGCTCTGAACTCATCTCTTCT |
| | | ACCTTCAAGAAGGAAAAGGCCAAAAGCTGTTCCATTATGTGAAGTGGAAATTGGAGGGGG |
| | | GAGTCGACCCCCTACTTCCTCCCTTGGGGTGCAGAGGCACGGGGAGAGGGAGGATGAGACA |
| | | ATTTAGGACACTGGACATGAGTTTTTCAGATGGCCACGGTGAGGGCTTGGAAGGAGACAGG |
| | | AATGGGGCGAGGAAGGAGCCAGGCCCGGCATGAGGACCTGACGCTGAGAGAGAACCATCA |
| | | TACCCCAAGCCAG |
| 23 | Homo | GTTCGTTGCAACAAATTGATGAGCAATGCTTTTTTATAATGCCAACTTTGTACAAAAAAGTT |
| | sapiens clone | GGCATGCCCTGGTCGTCCCGCGGCGCCCTCCTTCGGGACCTGGTCCTGGGCGTGCTGGGCAC |
| | ccsbBroadEn_03525 | CGCCGCCTTCCTGCTCGACCTGGGCACCGACCTGTGGGCCGCCGTCCAGTATGCGCTCGGCG |
| | XKR8 gene. | GCCGCTACCTGTGGGCGGCGCTGGTGCTGGCGCTGCTGGGCCTGGCCTCCGTGGCGCTGCA |
| | GenBank: | GCTCTTCAGCTGGCTCTGGCTGCGCGCTGACCCTGCCGGCCTGCACGGGTCGCAGCCCCCGC |
| | KJ894131.1 | GCCGCTGCCTGGCGCTGCTGCATCTCCTGCAGCTGGGTTACCTGTACAGGTGCGTGCAGGAG |
| | | CTGCGGCAGGGGCTGCTGGTGTGGCAGCAGGAGGAGCCCTCTGAGTTTGACTTGGCCTACG |
| | | CCGACTTCCTCGCCCTGGACATCAGCATGCTGCGGCTCTTCGAGACCTTCTTGGAGACGGCA |
| | | CCACAGCTCACGCTGGTGCTGGCCATCATGCTGCAGAGTGGCCGGGCTGAGTACTACCAGT |
| | | GGGTTGGCATCTGCACATCCTTCCTGGGCATCTCGTGGGCACTGCTCGACTACCACCGGGCC |
| | | TTGCGCACCTGCCTCCCCTCCAAGCCGCTCCTGGGCCTGGGCTCCTCCGTGATCTACTTCCTG |
| | | TGGAACCTGCTGCTGCTGTGGCCCCGAGTCCTGGCTGTGGCCCTGTTCTCAGCCCTCTTCCCC |
| | | AGCTATGTGGCCCTGCACTTCCTGGGCCTGTGGCTGGTACTGCTGCTCTGGGTCTGGCTTCA |
| | | GGGCACAGACTTCATGCCGGACCCCAGCTCCGAGTGGCTGTACCGGGTGACGGTGGCCACC |
| | | ATCCTCTATTTCTCCTGGTTCAACGTGGCTGAGGGCCGCACCCGAGGCCGGGCCATCATCCA |
| | | CTTCGCCTTCCTCCTGAGTGACAGCATTCTCCTGGTGGCCACCTGGGTGACTCATAGCTCCT |
| | | GGCTGCCCAGCGGGATTCCACTGCAGCTGTGGCTGCCTGTGGGATGCGGCTGCTTCTTTCTG |
| | | GGCCTGGCTCTGCGGCTTGTGTACTACCACTGGCTGCACCCTAGCTGCTGCTGGAAGCCCGA |
| | | CCCTGACCAGGTAGACGGGGCCCGGAGTCTGCTTTCTCCAGAGGGGTATCAGCTGCCTCAG |
| | | AACAGGCGCATGACCCATTTAGCACAGAAGTTTTTCCCCAAGGCTAAGGATGAGGCTGCTT |
| | | CGCCAGTGAAGGGATACCCAACTTTCTTGTACAAAGTTGGCATTATAAGAAAGCATTGCTTA |
| | | TCAATTTGTTGCAACGAAC |
| 24 | Homo | GTTCGTTGCAACAAATTGATGAGCAATGCTTTTTTATAATGCCAACTTTGTACAAAAAAGTT |
| | sapiens clone | GGCACCATGAAAAAGATGAGCAGGAATGTTTTGCTACAAATGGAGGAGGAGGAGGACGAC |
| | ccsbBroadEn_09792 | GACGATGGGGATATCGTGTTGGAAAACCTTGGACAGACAATTGTCCCCGATTTGGGATCAC |
| | ANO6 gene. | TGGAAAGTCAGCATGATTTTCGAACCCCGGAGTTTGAAGAATTTAATGGAAAACCTGACTC |
| | GenBank: | CCTCTTTTTTAATGATGGCCAGCGAAGAATTGACTTTGTTCTAGTATATGAGGATGAAAGCA |
| | KJ900398.1 | GAAAAGAGACCAATAAAAAGGGTACAAATGAAAAACAAAGGAGGAAAAGACAAGCATAC |
| | | GAATCTAACCTTATCTGTCATGGCCTGCAGTTAGAAGCAACAAGATCAGTATTGGATGACA |
| | | AGCTTGTATTTGTAAAAGTACACGCACCATGGGAGGTGTTATGTACGTATGCTGAGATAATG |
| | | CACATCAAATTGCCTCTGAAACCCAATGATCTGAAAAACCGGTCCTCAGCCTTTGGTACACT |
| | | CAACTGGTTTACCAAAGTCCTCAGTGTAGACGAAAGCATCATCAAGCCAGAGCAAGAGTTT |
| | | TTCACTGCCCCATTTGAGAAGAACCGGATGAATGATTTTTACATAGTTGATAGAGATGCTTT |
| | | CTTCAATCCAGCCACCAGAAGCCGCATTGTTTACTTCATCCTCTCTCGGGTCAAGTATCAAG |
| | | TGATAAACAATGTTAGCAAGTTTGGGATCAACAGACTTGTAAACTCTGGGATCTACAAGGC |
| | | AGCTTTCCCACTCCATGATTGCAAATTCCGCCGTCAGTCAGAGGATCCCAGCTGCCCTAATG |
| | | AACGGTACCTTCTGTACAGAGAATGGGCTCATCCTCGAAGCATATACAAAAAGCAGCCCTT |
| | | GGATCTTATCAGGAAATACTATGGAGAGAAGATTGGAATCTACTTTGCTTGGCTGGGCTATT |
| | | ACACTCAGATGCTTCTCCTGGCCGCAGTTGTAGGAGTGGCTTGCTTTCTCTATGGATATCTTA |
| | | ATCAAGATAACTGTACATGGAGCAAAGAAGTTTGTCATCCTGATATTGGTGGCAAGATCAT |
| | | AATGTGTCCTCAGTGTGATAGGCTTTGTCCATTCTGGAAACTCAATATTACTTGCGAGTCCT |
| | | CAAAGAAATTGTGCATCTTCGACAGTTTTGGAACCCTGGTCTTTGCAGTATTTATGGGAGTA |
| | | TGGGTTACCTTGTTTTTGGAGTTTTGGAAGCGACGCCAGGCAGAACTTGAGTATGAATGGGA |
| | | TACTGTTGAGTTACAGCAGGAAGAACAAGCCCGACCAGAATACGAAGCACGATGTACTCAC |
| | | GTAGTGATAAATGAGATTACTCAGGAAGAAGAACGCATTCCCTTTACTGCCTGGGGAAAAT |
| | | GTATACGGATAACCCTCTGTGCCAGTGCTGTCTTTTTCTGGATCCTATTGATCATCGCTTCAG |
| | | TTATTGGGATCATTGTCTATAGGCTCTCGGTGTTCATTGTATTTTCTGCAAAACTTCCCAAGA |
| | | ACATTAATGGAACAGACCCAATCCAGAAATACCTGACTCCACAGACAGCCACGTCCATCAC |
| | | GGCCTCCATCATCAGCTTTATAATTATCATGATTCTGAACACCATATATGAAAAAGTGGCAA |
| | | TTATGATTACTAACTTCGAACTCCCAAGGACCCAGACTGATTATGAGAACAGCCTCACCATG |
| | | AAGATGTTCTTATTCCAGTTTGTCAACTACTACTCTTCATGCTTCTACATAGCATTCTTTAAG |
| | | GGCAAATTTGTAGGCTATCCAGGAGACCCAGTTTATTGGTTGGGAAAATACAGAAATGAAG |
| | | AGTGTGACCCAGGTGGCTGTCTTCTTGAACTGACAACTCAGCTGACAATAATCATGGGAGG |
| | | AAAAGCAATCTGGAATAACATACAAGAAGTATTATTGCCCTGGATCATGAATCTAATTGGG |
| | | CGATTTCACAGAGTTTCTGGATCAGAAAAGATAACCCCACGATGGGAACAGGACTACCATC |
| | | TGCAGCCTATGGGCAAACTGGGATTATTTTATGAATATCTTGAAATGATTATTCAGTTTGGG |
| | | TTCGTCACCTTATTTGTGGCCTCTTTTCCACTGGCCCCTCTGTTGGCTCTCGTGAACAATATA |
| | | TTGGAAATAAGAGTGGACGCATGGAAACTGACCACCCAGTTTAGACGCCTGGTACCAGAGA |
| | | AAGCCCAAGACATTGGAGCATGGCAGCCCATCATGCAAGGAATAGCAATTCTGGCTGTGGT |
| | | GACCAATGCCATGATCATAGCTTTCACGTCGGACATGATCCCCCGCCTAGTGTACTACTGGT |
| | | CCTTCTCCGTCCCTCCCTACGGGGACCACACTTCCTACACCATGGAAGGGTACATCAACAAC |
| | | ACTCTCTCCATCTTCAAAGTCGCAGACTTCAAAAACAAAAGCAAGGGAAACCCGTACTCTG |
| | | ACCTGGGTAACCATACCACATGCAGGTATCGTGATTTCCGATACCCACCTGGACACCCCCAG |
| | | GAGTATAAACACAACATCTACTATTGGCATGTGATTGCAGCCAAGCTGGCTCTTATCATTGT |
| | | CATGGAGCACGTCATCTACTCTGTGAAATTTTTCATTTCATATGCAATTCCCGATGTATCAA |
| | | AACGCACAAAGAGCAAGATCCAGAGAGAAAAATACCTAACCCAAAAGCTTCTTCATGAGA |
| | | ATCACCTCAAAGATATGACGAAAAATATGGGGGTGATAGCTGAGCGGATGATAGAAGCAG |
| | | TAGATAACAATTTACGGCCAAAATCAGAATTGCCAACTTTCTTGTACAAAGTTGGCATTATA |
| | | AGAAAGCATTGCTTATCAATTTGTTGCAACGAAC |
| 25 | Homo | GCCTTCCGGAGCGTAGCGGCCTCTAGCTCGAGCAGCAGGAGCAGCCCGCACCGGACAACTT |
| | sapiens | GCGAGCCATGGGGCTGGCGGATGCGTCGGGACCGAGGGACACACAGGCACTGCTGTCTGCA |
| | solute carrier | ACACAAGCAATGGACCTGCGGAGGCGAGACTACCACATGGAACGGCCGCTGCTGAACCAG |
| | family 26 | GAGCATTTGGAGGAGCTGGGGCGCTGGGGCTCAGCACCTAGGACCCACCAGTGGCGGACCT |
| | member 6 | GGTTGCAGTGCTCCCGTGCTCGGGCCTATGCCCTTCTGCTCCAACACCTCCCGGTTTTGGTCT |
| | (SLC26A6). | GGTTACCCCGGTATCCTGTGCGTGACTGGCTCCTGGGTGACCTGTTATCCGGCCTGAGTGTG |
| | NCBI | GCCATCATGCAGCTTCCGCAGGGCTTGGCCTACGCCCTCCTGGCTGGATTGCCCCCCGTGTT |
| | Reference | TGGCCTCTATAGCTCCTTCTACCCTGTCTTCATCTACTTCCTGTTTGGCACTTCCCGGCACAT |
| | Sequence: | CTCCGTGGGGACCTTTGCTGTCATGTCTGTGATGGTGGGCAGTGTGACAGAATCCCTGGCCC |
| | NM_022911.3 | CGCAGGCCTTGAACGACTCCATGATCAATGAGACAGCCAGAGATGCTGCCCGGGTACAGGT |
| | | GGCCTCCACACTCAGTGTCCTGGTTGGCCTCTTCCAGGTGGGGCTGGGCCTGATCCACTTCG |
| | | GCTTCGTGGTCACCTACCTGTCAGAACCTCTTGTCCGAGGCTATACCACAGCTGCAGCTGTG |
| | | CAGGTCTTCGTCTCACAGCTCAAGTATGTGTTTGGCCTCCATCTGAGCAGCCACTCTGGGCC |
| | | ACTGTCCCTCATCTATACAGTGCTGGAGGTCTGCTGGAAGCTGCCCCAGAGCAAGGTTGGC |
| | | ACCGTGGTCACTGCAGCTGTGGCTGGGGTGGTGCTCGTGGTGGTGAAGCTGTTGAATGACA |
| | | AGCTGCAGCAGCAGCTGCCCATGCCGATACCCGGGGAGCTGCTCACGCTCATCGGGGCCAC |
| | | AGGCATCTCCTATGGCATGGGTCTAAAGCACAGATTTGAGGTAGATGTCGTGGGCAACATC |
| | | CCTGCAGGGCTGGTGCCCCCAGTGGCCCCCAACACCCAGCTGTTCTCAAAGCTCGTGGGCA |
| | | GCGCCTTCACCATCGCTGTGGTTGGGTTTGCCATTGCCATCTCACTGGGGAAGATCTTCGCC |
| | | CTGAGGCACGGCTACCGGGTGGACAGCAACCAGGAGCTGGTGGCCCTGGGCCTCAGTAACC |
| | | TTATCGGAGGCATCTTCCAGTGCTTCCCCGTGAGTTGCTCTATGTCTCGGAGCCTGGTACAG |
| | | GAGAGCACCGGGGGCAACTCGCAGGTTGCTGGAGCCATCTCTTCCCTTTTCATCCTCCTCAT |
| | | CATTGTCAAACTTGGGGAACTCTTCCATGACCTGCCCAAGGCGGTCCTGGCAGCCATCATCA |
| | | TTGTGAACCTGAAGGGCATGCTGAGGCAGCTCAGCGACATGCGCTCCCTCTGGAAGGCCAA |
| | | TCGGGCGGATCTGCTTATCTGGCTGGTGACCTTCACGGCCACCATCTTGCTGAACCTGGACC |
| | | TTGGCTTGGTGGTTGCGGTCATCTTCTCCCTGCTGCTCGTGGTGGTCCGGACACAGATGCCC |
| | | CACTACTCTGTCCTGGGGCAGGTGCCAGACACGGATATTTACAGAGATGTGGCAGAGTACT |
| | | CAGAGGCCAAGGAAGTCCGGGGGGTGAAGGTCTTCCGCTCCTCGGCCACCGTGTACTTTGC |
| | | CAATGCTGAGTTCTACAGTGATGCGCTGAAGCAGAGGTGTGGTGTGGATGTCGACTTCCTCA |
| | | TCTCCCAGAAGAAGAAACTGCTCAAGAAGCAGGAGCAGCTGAAGCTGAAGCAACTGCAGA |
| | | AAGAGGAGAAGCTTCGGAAACAGGCTGCCTCCCCCAAGGGCGCCTCAGTTTCCATTAATGT |
| | | CAACACCAGCCTTGAAGACATGAGGAGCAACAACGTTGAGGACTGCAAGATGATGCAGGT |
| | | GAGCTCAGGAGATAAGATGGAAGATGCAACAGCCAATGGTCAAGAAGACTCCAAGGCCCC |
| | | AGATGGGTCCACACTGAAGGCCCTGGGCCTGCCTCAGCCAGACTTCCACAGCCTCATCCTG |
| | | GACCTGGGTGCCCTCTCCTTTGTGGACACTGTGTGCCTCAAGAGCCTGAAGAATATTTTCCA |
| | | TGACTTCCGGGAGATTGAGGTGGAGGTGTACATGGCGGCCTGCCACAGCCCTGTGGTCAGC |
| | | CAGCTTGAGGCTGGGCACTTCTTCGATGCATCCATCACCAAGAAGCATCTCTTTGCCTCTGT |
| | | CCATGATGCTGTCACCTTTGCCCTCCAACACCCGAGGCCTGTCCCCGACAGCCCTGTTTCGG |
| | | TCACCAGACTCTGAACATGCTACATCCTGCCCAAGACTGCACCTCTGGAGGTGCAGGGCAC |
| | | CCTTGAGAAGCCCCTCACCCCTAGGCCGCCTCCAGGTGCTACCCAGGAGTCCCCTCCATGTA |
| | | CACACACACAACTCAGGGAAGGAGGTCCTGGGACTCCAAGTTCAGCGCTCCAGGTCTGGGA |
| | | CAGGGCCTGCATGCAGTCAGGCTGGCAGTGGCGCGGTACAGGGAGGGAACTGGTGCATATT |
| | | TTAGCCTCAGGAATAAAGATTTGTCTGCTCAA |
| 26 | Homo | GTTCGTTGCAACAAATTGATGAGCAATGCTTTTTTATAATGCCAACTTTGTACAAAAAAGTT |
| | sapiens clone | GGCATGACCTCACGTTTCCGATTGCCTGCTGGCAGAACCTACAATGTACGAGCATCAGAGTT |
| | ccsbBroadEn_01341 | GGCCCGAGACAGACAGCATACTGAAGTGGTTTGCAACATCCTTCTTCTGGATAACACTGTAC |
| | PTPN4 | AAGCTTTCAAAGTCAATAAACATGATCAGGGGCAAGTCTTGTTGGATGTCGTCTTCAAGCAT |
| | gene. | CTAGATTTGACTGAGCAGGACTATTTTGGTTTACAGTTGGCTGATGATTCCACAGATAACCC |
| | GenBank: | AAGGTGGCTGGATCCAAACAAACCAATAAGGAAGCAGCTAAAGAGAGGATCTCCTTACAG |
| | KJ891947.1 | TTTGAACTTTAGAGTCAAATTTTTTGTAAGTGACCCCAACAAGTTACAAGAAGAATATACAA |
| | | GGTACCAGTATTTTTTGCAAATTAAACAAGACATTCTTACTGGAAGATTACCCTGTCCTTCT |
| | | AATACTGCTGCCCTTTTAGCTTCATTTGCTGTTCAGTCTGAACTTGGAGACTACGATCAGTCA |
| | | GAGAACTTGTCAGGCTACCTCTCAGATTATTCTTTCATTCCTAATCAACCTCAAGATTTTGAA |
| | | AAAGAAATTGCAAAATTACATCAGCAACACATAGGCTTATCTCCTGCAGAAGCAGAATTTA |
| | | ATTACCTAAACACAGCACGTACCTTAGAACTCTATGGAGTTGAATTCCACTATGCAAGGGAT |
| | | CAGAGTAACAATGAAATTATGATTGGAGTGATGTCAGGAGGAATTCTGATTTATAAGAACA |
| | | GGGTACGAATGAATACCTTTCCATGGTTGAAGATTGTAAAAATTTCTTTTAAGTGCAAACAG |
| | | TTTTTTATTCAACTTAGAAAAGAATTGCATGAATCTAGAGAAACATTATTGGGATTTAATAT |
| | | GGTGAATTACAGAGCATGTAAAAATTTGTGGAAAGCATGTGTAGAACATCACACATTCTTC |
| | | CGTTTGGACAGACCACTTCCACCTCAAAAGAATTTTTTTGCACATTATTTTACATTAGGTTCA |
| | | AAATTCCGGTACTGTGGGAGAACTGAAGTCCAATCAGTTCAGTATGGCAAAGAAAAGGCAA |
| | | ATAAAGACAGGGTATTTGCAAGATCCCCAAGTAAGCCCTTGGCACGGAAATTAATGGATTG |
| | | GGAAGTAGTAAGCAGAAATTCAATATCTGATGACAGGTTAGAAACACAAAGTCTTCCATCA |
| | | CGATCTCCACCGGGAACTCCTAATCATCGAAATTCTACATTCACGCAGGAAGGAACCCGGT |
| | | TACGACCATCTTCAGTTGGTCATTTGGTAGACCATATGGTTCATACTTCCCCAAGCGAAGTG |
| | | TTTGTAAATCAGAGATCTCCGTCATCAACACAAGCTAATAGCATTGTTCTGGAATCATCACC |
| | | ATCACAAGAGACCCCTGGAGATGGGAAGCCTCCAGCTTTACCACCCAAACAGTCAAAGAAA |
| | | AACAGTTGGAACCAAATTCATTATTCACATTCGCAACAAGATCTAGAAAGTCATATTAATG |
| | | AAACATTTGATATTCCATCTTCTCCTGAAAAACCCACTCCTAATGGTGGTATTCCACATGAT |
| | | AATCTTGTCCTAATCAGAATGAAACCTGATGAAAATGGGAGGTTTGGATTCAATGTAAAGG |
| | | GAGGATATGATCAGAAGATGCCTGTGATTGTGTCTCGAGTAGCACCAGGAACACCTGCTGA |
| | | CCTCTGTGTCCCTAGACTGAATGAAGGGGACCAAGTTGTACTGATCAATGGTCGGGACATT |
| | | GCAGAACACACTCATGATCAGGTTGTGCTGTTTATTAAAGCTAGTTGTGAGAGACATTCTGG |
| | | GGAACTCATGCTTCTAGTTCGACCTAATGCTGTATATGATGTAGTGGAAGAAAAGCTAGAA |
| | | AATGAGCCAGATTTCCAGTATATTCCTGAGAAAGCCCCACTAGATAGTGTGCATCAGGATG |
| | | ACCATTCCCTGCGGGAGTCAATGATCCAGCTAGCTGAGGGGCTTATCACTGGAACAGTCCT |
| | | GACACAGTTTGATCAACTGTATCGGAAAAAACCTGGAATGACAATGTCCTGTGCCAAATTA |
| | | CCTCAGAATATTTCCAAAAATAGATACAGAGATATTTCGCCTTATGATGCCACACGGGTCAT |
| | | TTTAAAAGGTAATGAAGACTACATCAATGCGAACTATATAAATATGGAAATTCCTTCTTCCA |
| | | GCATTATAAATCAGTACATTGCTTGTCAAGGGCCATTACCACACACTTGTACAGATTTTTGG |
| | | CAGATGACTTGGGAACAAGGCTCCTCTATGGTTGTAATGTTGACCACACAAGTTGAACGTG |
| | | GCAGAGTTAAATGTCACCAATATTGGCCAGAACCCACAGGCAGTTCATCTTATGGATGCTA |
| | | CCAAGTTACCTGCCACTCTGAAGAAGGAAACACTGCCTATATCTTCAGGAAGATGACCCTA |
| | | TTTAACCAAGAGAAAAATGAAAGTCGTCCACTCACTCAGATCCAGTACATAGCCTGGCCTG |
| | | ACCATGGAGTCCCTGATGATTCGAGTGACTTTCTAGATTTTGTTTGTCATGTACGAAACAAG |
| | | AGGGCTGGCAAGGAAGAACCCGTTGTTGTCCATTGCAGTGCTGGAATCGGAAGAACTGGGG |
| | | TTCTTATTACTATGGAAACAGCCATGTGTCTCATTGAATGCAATCAGCCAGTTTATCCACTA |
| | | GATATTGTAAGAACAATGAGAGATCAGCGAGCCATGATGATCCAAACACCTAGTCAATACA |
| | | GATTTGTATGTGAAGCTATTTTGAAAGTTTATGAAGAAGGCTTTGTTAAACCCTTAACAACA |
| | | TCAACAAATAAATACCCAACTTTCCTTGTACAAAGTTGGCATTATAAGAAAGCATTGCTTAT |
| | | CAATTTGTTGCAACGAAC |
| 27 | Homo | GTTCGTTGCAACAAATTGATGAGCAATGCTTTTTTATAATGCCAACTTTGTACAAAAAAGTT |
| | sapiens clone | GGCATGGTGAACCACAGTCTTCACCCTACTGAACCCGTCAAGGTCACTCTGCCAGACGCCTT |
| | ccsbBroadEn_12539 | TTTGCCTGCTCAAGTCTGTAGTGCCAGGATTCAGGAAAATGGCTCCCTTATCACCATCCTGG |
| | ATG9A | TCATTGCTGGTGTCTTCTGGATCCACCGGCTTATCAAGTTCATCTATAACATTTGCTGCTACT |
| | gene. | GGGAGATCCACTCCTTCTACCTGCACGCTCTGCGCATCCCTATGTCTGCCCTTCCGTATTGCA |
| | GenBank: | CGTGGCAAGAAGTGCAGGCCCGGATCGTGCAGACGCAGAAGGAGCACCAGATCTGCATCC |
| | KJ903145.1 | ACAAACGTGAGCTGACAGAACTGGACATCTACCACCGCATCCTCCGTTTCCAGAACTACAT |
| | | GGTGGCACTGGTTAACAAATCCCTCCTGCCTCTGCGCTTCCGCCTGCCTGGCCTCGGGGAAG |
| | | CTGTCTTCTTCACCCGTGGTCTCAAGTACAACTTTGAGCTGATCCTCTTCTGGGGACCTGGCT |
| | | CTCTGTTTCTCAATGAATGGAGCCTCAAGGCCGAGTACAAACGTGGGGGGCAACGGCTAGA |
| | | GCTGGCCCAGCGCCTCAGCAACCGCATCCTGTGGATTGGCATCGCTAACTTCCTGCTGTGCC |
| | | CCCTCATCCTCATATGGCAAATCCTCTATGCCTTCTTCAGCTATGCTGAGGTGCTGAAGCGG |
| | | GAGCCGGGGGCCCTGGGAGCACGCTGCTGGTCACTCTATGGCCGCTGCTACCTCCGCCACTT |
| | | CAACGAGCTGGAGCACGAGCTGCAGTCCCGCCTCAACCGTGGCTACAAGCCCGCCTCCAAG |
| | | TACATGAATTGCTTCTTGTCACCTCTTTTGACACTGCTGGCCAAGAATGGAGCCTTCTTCGCT |
| | | GGCTCCATCCTGGCTGTGCTTATTGCCCTCACCATTTATGACGAAGATGTGTTGGCTGTGGA |
| | | ACATGTGCTGACCACCGTCACACTCCTGGGGGTCACCGTGACCGTGTGCAGGTCCTTTATCC |
| | | CGGACCAGCACATGGTGTTCTGCCCTGAGCAGCTGCTCCGCGTGATCCTCGCTCACATCCAC |
| | | TACATGCCTGACCACTGGCAGGGTAATGCCCACCGCTCGCAGACCCGGGACGAGTTTGCCC |
| | | AGCTCTTCCAGTACAAGGCAGTGTTCATTTTGGAAGAGTTGCTGAGCCCCATTGTCACACCC |
| | | CTCATCCTCATCTTCTGCCTGCGCCCACGGGCCCTGGAGATTATAGACTTCTTCCGAAACTTC |
| | | ACCGTGGAGGTCGTTGGTGTGGGAGATACCTGCTCCTTTGCTCAGATGGATGTTCGCCAGCA |
| | | TGGTCATCCCCAGTGGCTATCTGCTGGGCAGACAGAGGCCTCAGTGTACCAGCAAGCTGAG |
| | | GATGGAAAGACAGAGTTGTCACTCATGCACTTTGCCATCACCAACCCTGGCTGGCAGCCAC |
| | | CACGTGAGAGCACAGCCTTCCTAGGCTTCCTCAAGGAGCAGGTTCAGCGGGATGGAGCAGC |
| | | TGCTAGCCTCGCCAAGGGGGTCTGCTCCCTGAAAATGCCCTCTTTACGTCTATCCAGTCCTT |
| | | ACAATCTGAGTCTGAGCCCCTGCCCAACTTTCTTGTACAAAGTTGGCATTATAAGAAAGCAT |
| | | TGCTTATCAATTTGTTGCAACGAAC |
| 28 | Homo | GTTCGTTGCAACAAATTGATGAGCAATGCTTTTTTATAATGCCAACTTTGTACAAAAAAGTT |
| | sapiens clone | GGCATGCCGTCGGAGAAGACCTTCAAGCAGCGCCGCACCTTCGAACAAAGAGTAGAAGAT |
| | ccsbBroadEn_04252 | GTCCGACTTATTCGAGAGCAGCATCCAACCAAAATCCCGGTGATAATAGAACGATACAAGG |
| | MAP1LC3B | GTGAGAAGCAGCTTCCTGTTCTGGATAAAACAAAGTTCCTTGTACCTGACCATGTCAACATG |
| | gene. | AGTGAGCTCATCAAGATAATTAGAAGGCGCTTACAGCTCAATGCTAATCAGGCCTTCTTCCT |
| | GenBank: | GTTGGTGAACGGACACAGCATGGTCAGCGTCTCCACACCAATCTCAGAGGTGTATGAGAGT |
| | KJ894858. 1 | GAGAAAGATGAAGATGGATTCCTGTACATGGTCTATGCCTCCCAGGAGACGTTCGGGATGA |
| | | AATTGTCAGTGTACCCAACTTTCTTGTACAAAGTTGGCATTATAAGAAAGCATTGCTTATCA |
| | | ATTTGTTGCAACGAAC |
| 29 | Homo | GTTCGTTGCAACAAATTGATGAGCAATGCTTTTTTATAATGCCAACTTTGTACAAAAAAGTT |
| | sapiens clone | GGCATGAAGGTTGTCCCAGAAAAGAATGCTGTCCGGATACTCTGGGGGCGAGAACGGGGTG |
| | ccsbBroadEn_08557 | CTCGGGCCATGGGAGCTCAGCGGCTTCTGCAGGAGCTGGTAGAGGATAAAACCCGGTGGAT |
| | AMBRA1 | GAAATGGGAGGGCAAGAGAGTAGAACTGCCGGATAGTCCACGCTCTACCTTCTTATTGGCC |
| | gene. | TTCAGCCCAGACAGGACTCTCTTAGCCTCCACCCATGTGAACCATAATATCTATATTACGGA |
| | GenBank: | GGTGAAGACTGGCAAGTGTGTTCATTCCCTGATTGGACACCGCCGTACTCCATGGTGTGTCA |
| | KJ899163.1 | CTTTTCATCCCACCATCTCAGGCCTTATTGCTTCTGGCTGCCTAGATGGGGAGGTTAGGATTT |
| | | GGGATTTACACGGTGGCAGTGAAAGCTGGTTCACAGATAGCAACAATGCCATTGCCTCCCT |
| | | GGCTTTCCACCCTACGGCTCAGCTCCTGCTGATTGCCACTGCCAATGAGATCCACTTCTGGG |
| | | ACTGGAGTCGACGGGAACCCTTTGCTGTGGTGAAGACAGCTAGTGAGATGGAACGGGTCCG |
| | | TCTGGTGAGATTTGATCCACTTGGACACTACTTACTCACAGCAATTGTTAACCCCTCTAATC |
| | | AACAGGGTGATGACGAACCAGAGATCCCCATAGATGGAACAGAATTATCCCACTACCGTCA |
| | | GCGTGCCCTCCTGCAATCACAGCCAGTTCGCCGGACGCCTCTCCTCCACAATTTCCTGCACA |
| | | TGCTGTCCTCCCGCTCTTCTGGCATCCAGACCGAGCCCTTCCATCCCCCGGAGCAGGCCTCG |
| | | TCAACGCAGCAGGACCAGGGCCTCCTGAACCGGCCGTCTGCCTTCAGTACAGTCCAGAGCA |
| | | GCACTGCCGGCAACACGCTCCGCAACCTCAGTCTGGGTCCTACCCGCCGCTCTTTGGGAGG |
| | | GCCTCTGTCTAGCCACCCTTCTAGGTATCACCGAGAAATAGCTCCTGGGTTGACAGGATCTG |
| | | AGTGGACCCGGACAGTACTCAGTCTGAACTCCCGCTCTGAGGCGGAATCCATGCCCCCGCC |
| | | CAGAACCAGTGCCTCTTCGGTGAGTTTGCTGTCTGTGCTGAGACAGCAGGAAGGTGGCTCTC |
| | | AGGCATCTGTGTACACTTCAGCCACAGAAGGGAGGGGTTTTCCGGCATCAGGGTTGGCAAC |
| | | TGAGTCAGATGGAGGGAATGGCTCCAGCCAAAACAACTCGGGCAGCATTCGCCATGAGCTT |
| | | CAGTGTGACCTGAGACGCTTCTTTCTGGAGTATGACCGGCTTCAGGAGCTGGATCAGAGCCT |
| | | GAGTGGGGAAGCTCCCCAGACCCAACAGGCCCAGGAAATGCTCAACAATAACATTGAATCT |
| | | GAGAGGCCAGGCCCTTCCCACCAGCCCACCCCACACAGCAGTGAGAACAACTCCAACCTGT |
| | | CCCGTGGCCACCTGAATCGCTGTCGTGCTTGCCACAATCTCCTGACCTTCAACAACGATACC |
| | | CTGCGCTGGGAAAGAACCACACCTAACTACTCCTCTGGCGAGGCTAGTTCCTCTTGGCAGGT |
| | | CCCCAGCTCCTTTGAGAGTGTGCCATCAAGTGGCAGCCAGTTGCCACCTCTCGAGCGGACTG |
| | | AGGGCCAAACGCCCAGCTCCAGCAGGCTGGAGTTGAGCAGCTCTGCTAGTCCGCAGGAGGA |
| | | GAGGACTGTGGGGGTGGCCTTTAACCAGGAGACAGGCCACTGGGAAAGAATTTACACCCAG |
| | | TCCAGCAGATCTGGAACTGTGTCACAGGAGGCCTTACATCAGGATATGCCTGAGGAGAGCT |
| | | CTGAGGAGGATTCACTCAGGAGGAGGCTGCTGGAATCTTCCCTCATTTCATTATCCCGTTAT |
| | | GATGGAGCAGGATCCAGAGAGCACCCAATTTACCCAGACCCAGCGAGATTATCTCCTGCTG |
| | | CATACTACGCCCAGAGGATGATCCAGTATCTCTCACGGAGAGACAGTATTCGCCAGCGCTC |
| | | CATGCGCTACCAACAGAACCGTCTCCGTTCTTCCACCTCCTCCTCTTCCTCAGACAACCAGG |
| | | GTCCATCAGTAGAGGGAACCGACTTGGAATTTGAGGACTTTGAGGACAATGGTGACAGATC |
| | | CAGGCACCGAGCTCCACGCAATGCCCGGATGTCTGCACCTTCGCTTGGACGCTTTGTCCCAA |
| | | GGCGTTTCTTGCTGCCTGAGTACTTGCCTTATGCTGGGATTTTTCATGAACGTGGACAGCCT |
| | | GGCTTGGCTACTCACTCTTCTGTTAACAGGGTCCTGGCAGGGGCAGTGATCGGTGATGGACA |
| | | GTCTGCTGTGGCCAGTAACATTGCCAATACTACCTACCGGCTCCAGTGGTGGGACTTCACTA |
| | | AGTTTGACCTCCCTGAAATCAGTAATGCTTCCGTGAATGTGCTGGTGCAGAACTGCAAGATC |
| | | TACAATGATGCCAGCTGTGACATTTCTGCAGATGGCCAGCTCCTGGCAGCTTTCATCCCCAG |
| | | CAGCCAGAGGGGCTTTCCTGATGAAGGCATCCTGGCAGTGTACTCCCTGGCCCCCCATAACC |
| | | TGGGCGAAATGCTCTACACCAAGCGATTTGGTCCCAATGCCATTTCGGTGAGCCTGTCCCCA |
| | | ATGGGCAGATATGTAATGGTGGGCTTGGCCTCACGAAGGATCCTGCTGCACCCCTCCACAG |
| | | AGCACATGGTGGCCCAGGTCTTCAGGCTGCAACAGGCCCATGGTGGAGAGACCTCCATGAG |
| | | GAGAGTTTTCAACGTCCTTTATCCCATGCCTGCCGACCAGCGGAGACATGTCAGTATCAACT |
| | | CTGCCCGTTGGCTGCCTGAGCCAGGGCTTGGCTTGGCCTATGGTACTAACAAAGGAGACCT |
| | | GGTGATCTGCCGACCAGAGGCCTTAAACTCTGGTGTTGAGTACTACTGGGACCAGCTGAAC |
| | | GAGACGGTCTTCACTGTCCATTCCAACAGCAGGAGCAGCGAGCGGCCTGGAACCAGCAGAG |
| | | CCACATGGAGGACAGACAGAGACATGGGGCTGATGAATGCCATTGGGCTTCAGCCCCGGAA |
| | | CCCTGCCACCTCAGTGACATCTCAGGGCACCGAGACTCTGGCCCTTCAGCTGCAGAATGCC |
| | | GAAACACAGACTGAGAGGGAGGTGCCGGAGCCAGGGACAGCCGCCTCAGGTCCTGGTGAA |
| | | GGTGAGGGTTCAGAGTATGGTGCCAGTGGAGAAGATGCGCTCAGCAGGATCCAGAGGCTG |
| | | ATGGCGGAGGGCGGCATGACAGCCGTGGTGCAGCGGGAGCAGAGCACCACCATGGCCTCC |
| | | ATGGGCGGCTTCGGCAACAACATCATCGTCAGCCACCGCATTCACCGCAGCTCTCAGACGG |
| | | GCACTGAACCTGGTGCCGCCCACACCTCCTCACCCCAGCCCTCCACCTCTCGGGGACTGCTC |
| | | CCAGAGGCCGGGCAACTGGCAGAGCGAGGCCTAAGCCCCCGGACAGCTTCCTGGGACCAG |
| | | CCTGGTACCCCTGGGCGGGAGCCAACCCAGCCAACCCTGCCCTCTTCCTCCCCTGTCCCCAT |
| | | TCCTGTTTCCCTTCCCAGCGCTGAGGGACCAACCGTCCACTGCGAGTTGACCAATAACAACC |
| | | ACCTTCTGGATGGTGGCAGCAGCAGGGGGGACGCTGCAGGCCCTAGGGGAGAACCACGGA |
| | | ACAGGTACCCAACTTTCTTGTACAAAGTTGGCATTATAAGAAAGCATTGCTTATCAATTTGT |
| | | TGCAACGAAC |
| 30 | Homo | GACTGATTTCGAGTTTCCGGTCAGGTTAGGCCGGGGGGGTGCGGTCCTGGTCGGAAGGAGG |
| | sapiens | TGGAGAGTCGGGGGTCACCAGGCCTATCCTTGGCGCCACAGTCGGCCACCGGGGCTCGCCG |
| | mitochondria | CCGTCATGGAGAGCGGAGGGCGGCCCTCGCTGTGCCAGTTCATCCTCCTGGGCACCACCTCT |
| | 1 E3 | GTGGTCACCGCCGCCCTGTACTCCGTGTACCGGCAGAAGGCCCGGGTCTCCCAAGAGCTCA |
| | ubiquitin | AGGGAGCTAAAAAAGTTCATTTGGGTGAAGATTTAAAGAGTATTCTTTCAGAAGCTCCAGG |
| | protein ligase | AAAATGCGTGCCTTATGCTGTTATAGAAGGAGCTGTGCGGTCTGTTAAAGAAACGCTTAAC |
| | 1 (MUL1). | AGCCAGTTTGTGGAAAACTGCAAGGGGGTAATTCAGCGGCTGACACTTCAGGAGCACAAGA |
| | NCBI | TGGTGTGGAATCGAACCACCCACCTTTGGAATGATTGCTCAAAGATCATTCATCAGAGGAC |
| | Reference | CAACACAGTGCCCTTTGACCTGGTGCCCCACGAGGATGGCGTGGATGTGGCTGTGCGAGTG |
| | Sequence: | CTGAAGCCCCTGGACTCAGTGGATCTGGGTCTAGAGACTGTGTATGAGAAGTTCCACCCCTC |
| | NM_024544.3 | GATTCAGTCCTTCACCGATGTCATCGGCCACTACATCAGCGGTGAGCGGCCCAAAGGCATC |
| | | CAAGAGACCGAGGAGATGCTGAAGGTGGGGGCCACCCTCACAGGGGTTGGCGAACTGGTC |
| | | CTGGACAACAACTCTGTCCGCCTGCAGCCGCCCAAACAAGGCATGCAGTACTATCTAAGCA |
| | | GCCAGGACTTCGACAGCCTGCTGCAGAGGCAGGAGTCGAGCGTCAGGCTCTGGAAGGTGCT |
| | | GGCGCTGGTTTTTGGCTTTGCCACATGTGCCACCCTCTTCTTCATTCTCCGGAAGCAGTATCT |
| | | GCAGCGGCAGGAGCGCCTGCGCCTCAAGCAGATGCAGGAGGAGTTCCAGGAGCATGAGGC |
| | | CCAGCTGCTGAGCCGAGCCAAGCCTGAGGACAGGGAGAGTCTGAAGAGCGCCTGTGTAGTG |
| | | TGTCTGAGCAGCTTCAAGTCCTGCGTCTTTCTGGAGTGTGGGCACGTTTGTTCCTGCACCGA |
| | | GTGCTACCGCGCCTTGCCAGAGCCCAAGAAGTGCCCTATCTGCAGACAGGCGATCACCCGG |
| | | GTGATACCCCTGTACAACAGCTAATAGTTTGGAAGCCGCACAGCTTGACCTGGAAGCACCC |
| | | CTGCCCCCTTTTCAGGGATTTTTATCTCGAGGCCTTTGGAGGAGCAGTGGTGGGGGTAGCTG |
| | | TCACCTCCAGGTATGATTGAGGGAGGAATTGGGTAGAAACTCTCCAGACCCATGCCTCCAA |
| | | TGGCAGGATGCTGCCTTTCCCACCTGAGAGGGGACCCTGTCCATGTGCAGCCTCATCAGAGC |
| | | CTCACCCTGGGAGGATGCCGTGGCGTCTCCTCCCAGGAGCCAGATCAGTGCGAGTGTGACT |
| | | GAAAATGCCTCATCACTTAAGCACCAAAGCCAGTGATCAGCAGCTCTTCTGTTCCTGTGTCT |
| | | TCTGTTTTTTTCTGGTGAATCGTTGCTTGCTGTGGACTTGGTGGAGGACTCAGAGGGGAGGA |
| | | AAGGCTGGGCCCCGAGTACAACGGATGCCTTGGGTGCTGCCTCCGAAGAGACTCTGCCGCA |
| | | GCTTTTCTTCTTTTTCCTCATGCCCCGGGAAACAGTCTTTCTTCAGAATTGTCAGGCTGGGCA |
| | | GGTCAACTTGTGTTCCTTTCCCCTCACCTGCTTGCCTCCTTAACGCCTGCACGTGTGTGTAGA |
| | | GGACAAAAGAAAGTGAAGTCAGCACATCCGCTTCTGCCCAGATGGTCGGGGCCCCGGGCAA |
| | | CAGATTGAAGAGAGATCATGTGAAGGGCAGTTGGTCAGGCAGGCCTCCTGGTTTCGCCACT |
| | | GGCCCTGATTTGAACTCCTGCCACTTGGGAGAGCTCGGGGTGGTCCCTGGTTTTCCCTCCTG |
| | | GAGAATGAGGCGCAGAGGCCTCGCCTCCTGAAGGACGCAGTGTGGATGCCACTGGCCTAGT |
| | | GTCCTGGCCTCACAGCTTCCTTGCAAGGCTGTCACAAGGAAAAGCAGCCGGCTGGCACCCT |
| | | GAGCATATGCCCTCTTGGGGCTCCCTCATCCAGCCCGTCGCAGCTTTGACATCTTGGTGTAC |
| | | TCATGTCGCTTCTCCTTGTGTTACCCCCTCCCAGTATTACCATTTGCCCCTCACCTGCCCTTG |
| | | GTGAGCCTTTTAGTGCAAGACAGATGGGGCTGTTTTCCCCCACCTCTGAGTAGTTGGAGGTC |
| | | ACATACACAGCTCTTTTTTTATTGCCCTTTTCTGCCTCTGAATGTTCATCTCTCGTCCTCCTTT |
| | | GTGCAGGCGAGGAAGGGGTGCCCTCAGGGGCCGACACTAGTATGATGCAGTGTCCAGTGTG |
| | | AACAGCAGAAATTAAACATGTTGCAACCAA |
| 31 | Homo | GCGCTGCCCCATACCTGAAGACCAAGTTTATCTGTGTGACACCGTAAGTGGCTTCCTTTCCC |
| | sapiens | CGTTTTGCCTTCATTTCTAATATCCTCAGTTATCCCTGGGAATGGGACACTGGGTGAGAGTT |
| | STAT3 | AATCTGCCAAAGGTTGGAAGCCCCTGGGCTATGTTTAGTACTCAAAGTGACCTTGTGTGTTT |
| | (STAT3) | AAAAAGCTTGAGCTTTTATTTTTCTGTTGGAGACCAGAGTTTGATGGCTTGTGTGTGTGTGTT |
| | gene. | TTGTTCTTTTTTTTTTTTCCATTGTGTCTTGTCAACCCCCCGTTTCCCCTCCTGCTGCCCCCCA |
| | GenBank: | TTTCCTACAGAACGACCTGCAGCAATACCATTGACCTGCCGATGTCCCCCCGCACTTTAGAT |
| | AF332508.1 | TCATTGATGCAGTTTGGAAATAAKGGTGAAGGTGCTGAACCCTCAGCAGGAGGGCAGTTTG |
| 32 | Homo | ATGCTTTTGAGCCAGAATGCCTTCATCTTCAGATCACTTAATTTGGTTCTCATGGTGTATATC |
| | sapiens full | AGCCTCGTGTTTGGTATTTCATATGATTCGCCTGATTACACAGATGAATCTTGCACTTTCAAG |
| | open reading | ATATCATTGCGAAATTTCCGGTCCATCTTATCATGGGAATTAAAAAACCACTCCATTGTACC |
| | frame cDNA | AACTCACTATACATTGCTGTATACAATCATGAGTAAACCAGAAGATTTGAAGGTGGTTAAG |
| | clone | AACTGTGCAAATACCACAAGATCATTTTGTGACCTCACAGATGAGTGGAGAAGCACACACG |
| | RZPDo834C | AGGCCTATGTCACCGTCCTAGAAGGATTCAGCGGGAACACAACGTTGTTCAGTTGCTCACA |
| | 0132D for | CAATTTCTGGCTGGCCATAGACATGTCTTTTGAACCACCAGAGTTTGAGATTGTTGGTTTTA |
| | gene | CCAACCACATTAATGTGATGGTGAAATTTCCATCTATTGTTGAGGAAGAATTACAGTTTGAT |
| | IFNAR2, | TTATCTCTCGTCATTGAAGAACAGTCAGAGGGAATTGTTAAGAAGCATAAACCCGAAATAA |
| | interferon | AAGGAAACATGAGTGGAAATTTCACCTATATCATTGACAAGTTAATTCCAAACACGAACTA |
| | (alpha, beta | CTGTGTATCTGTTTATTTAGAGCACAGTGATGAGCAAGCAGTAATAAAGTCTCCCTTAAAAT |
| | and omega) | GCACCCTCCTTCCACCTGGCCAGGAATCAGAATCAGCAGAATCTGCCAAAATAGGAGGAAT |
| | receptor 2. | AATTACTGTGTTTTTGATAGCATTGGTCTTGACAAGCACCATAGTGACACTGAAATGGATTG |
| | GenBank: | GTTATATATGCTTAAGAAATAGCCTCCCCAAAGTCTTGAGGCAAGGTCTCGCTAAGGGCTG |
| | CR541817.1 | GAATGCAGTGGCTATTCACAGGTGCAGTCATAATGCACTACAGTCTGAAACTCCTGAGCTC |
| | | AAACAGTCGTCCTGCCTAAGCTTCCCCAGTAGCTGGGATTACAAGCGTGCATCCCTGTGCCC |
| | | CAGTGATTAA |
| 33 | Homo | AGTGGGAAAGGAGAGGAGAGGGACTATACTTCCTCCTCCCTGGGGCCCCCTGCAGAGCATC |
| | sapiens | TGGGAAGCAAGGCTTCCCTACATCCTCCATGCACCCCCTTAGAGTTTTCAATTCCTTTCCTCG |
| | STAT5A | TGATCCTGCCAACTAAGACACTGTGACCACACAGAGAAGGTGGGGAGAACGCAGACATTTT |
| | gene for | GGCTTCTGCAGCTTTGAAGTTCTTTTTTTTTTTTTCCTCTGAAGTTAAAAGAATGAAACTGGG |
| | signal | AGAGGTAGTAAGGGGCAAGAAAGGAGAGTGGAAATGGAGAGAAAAGGGCAGCTCTGAGA |
| | transducer | AGCAGCTGGGGAGGGAGGCAGATGAGAATGCACCCCCCCCAACAGAACATGCAGTCTTGG |
| | and activator | CCCAGCTGTGCTGTGAGTGGGCAGCTGGGCTGGCCCCTCCTCTGGTGCTGCCAACCCGCTGC |
| | of | CAGGCAGAGGGGAGGCCCAGAGGAGAGGGAAGCTGGGCAAAGGGGATGGAAGGCGTCCA |
| | transcription | GCCCGACCTTACCAAACCCCTTGGGCCTCGTGGGAAGGGGCCTCTTGGAGAGGGGGACTGA |
| | 5A. | GGCTCTAGACAGGATATTCACTGCTGTGGCAAGGCCTGTAGAGAGTTTCGAAGTTAGGAGG |
| | GenBank: | ACTCAAGACGGTCCCTCCCTGGACTTTTCTGAAGGTAGAACCAGCCTCATAAGTAACTAGGC |
| | AJ412877.1 | TGGGTGAACGGGGGCGCTGGCTAGTTTATGGATCACAGTCGGCTGGTGAGGCCACGTGCCT |
| | | ACTGTGTGGCCCTGGGTGGCCCCGGGCAAGCCCCTTTCCCTCTCAGGACTTCCATTTCTTAC |
| | | CTGCAAAATTGTGGAGAGGGGGAGGGCTGAAACACATGACTGCCAAGATTCTTTCCAGTTC |
| | | CTCCGTCAGGGTTGAGTTTAGATGGCCGGAGTAAAAGAAGGAGGGAGGTGCTGCGGTGGTG |
| | | GGGGTGATCTTGGCTTCACTAGAATCCCCAGTTCTTCCCCTCTCTACAGTTTTGTCTCTGAGG |
| | | TCACAAAACCTGTGGCCCCCAAGACACACATGCGCACACACGCGCGTGCACACACACACCC |
| | | CACACATTTATTTTTTAATCTAGGGGCTCAAAAGATGACACGCGCCAGAGCTGGAAGGCGT |
| | | CGCCAATTGGTCCACTTTTCCCTCCTCCCTTTTTGCGGATGAGAAAACTGAGGCCCAGGTTT |
| | | GGGATTTCCAGAGCCCGGGATTTCCCGGCAACGCCCGACAACCACATTCCCCCGGCTATTCT |
| | | GACCCGCCCCGGTTCCGGGACGCTCCCTGGGAGCCGCCGCCGAGGGCCTGCTGGGACTCCC |
| | | GGGGGACCCCGCCGTCGGGGCAGCCCCCACGCCCGGCGCCGCCCGCCGGGAACGGCCGCC |
| | | GCTGTTGCGCACTTGCAGGGGAGCCGGCGACTGAGGGCGAGGCAGGGAGGGAGCAAGCGG |
| | | GGCTGGGAGGGCTGCTGGCGCGGGCTCGCGCGCTGTGTATGGTCTATCGCAGGCAGCTGAC |
| | | CTTTGAGGAGGAAATCGCTGCTCTCCGCTCCTTCCTGTAGTAACAGCCGCCGCTGCCGCCGC |
| | | CGCCAGGAACCCCGGCCGGGAGCGAGAGCCGCGGGGCGCAGAGCCGGCCCGGCTGCCGGA |
| | | CGGTGCGGCCCCACCAGGTGGGTGACCCGGTGGCGCGTCCTCGGCGGCGCGCCGAGAGGG |
| | | GACACTCTACTGCCGCCCCGGCACCTCCGGCACCCGGAGCTCGACGGCCGGGCGCAGCGCG |
| | | GGGATCAGTCCCCGGCACTGCGAGGGAGTTGGCCCAGCGCAGACGAGGGACGGGGGGACG |
| | | TGGGGACAGGGGTCGGGGATGAAAGGCAGAGGCCAGGGAGGGCGCCGTCCTGGCACGCCT |
| | | CGGAGAGGGAGCACCTGTCGGGCTGGACCCGGGGAGGCACTAGTGCACAGATGGGGGAGC |
| | | AGCCGAAGAGGGGAGCGCCCAAGTC |
| 34 | Homo | GTTCGTTGCAACAAATTGATGAGCAATGCTTTTTTATAATGCCAACTTTGTACAAAAAAGTT |
| | sapiens clone | GGCATGGAAGGGAGAGGACCGTACCGGATCTACGACCCTGGGGGCAGCGTGCCCTCAGGA |
| | ccsbBroadEn_15702 | GAGGCATCCGCAGCTTTTGAGCGCCTAGTGAAGGAGAATTCCCGGCTGAAGGAAAAAATGC |
| | TNIP1 gene. | AAGGGATAAAGATGTTAGGGGAGCTTTTGGAAGAGTCCCAGATGGAAGCGACCAGGCTCC |
| | GenBank: | GGCAGAAGGCAGAGGAGCTAGTGAAGGACAACGAGCTGCTCCCACCACCTTCTCCCTCCTT |
| | KJ906032.1 | GGGCTCCTTCGACCCCCTGGCTGAGCTCACAGGAAAGGACTCAAATGTCACAGCATCTCCC |
| | | ACAGCCCCTGCATGCCCCAGTGACAAGCCAGCACCAGTCCAGAAGCCTCCATCCAGTGGCA |
| | | CCTCCTCTGAATTTGAAGTGGTCACTCCTGAGGAGCAGAATTCACCAGAGAGCAGCAGCCA |
| | | TGCCAATGCGATGGCGCTGGACCCCCTGCCCCGTGAGGACGGCAACCTGATGCTGCACCTG |
| | | CAGCGCCTGGAGACCACGCTGAGTGTGTGTGCCGAGGAGCCGGACCACGGCCAGCTCTTCA |
| | | CCCACCTGGGCCGCATGGCCCTGGAGTTCAACCGACTGGCATCCAAGGTGCACAAGAATGA |
| | | GCAGCGCACCTCCATTCTGCAGACCCTGTGTGAGCAGCTTCGGAAGGAGAACGAGGCTCTG |
| | | AAGGCCAAGTTGGATAAGGGCCTGGAACAGCGGGATCAGGCTGCCGAGAGGCTGCGGGAG |
| | | GAAAATTTGGAGCTCAAGAAGTTGTTGATGAGCAATGGCAACAAAGAGGGTGCGTCTGGGC |
| | | GGCCAGGCTCACCGAAGATGGAAGGGACAGGCAAGAAGGCAGTGGCTGGACAGCAGCAGG |
| | | CTAGTGTGACGGCAGGTAAGGTCCCAGAGGTGGTGGCCTTGGGCGCAGCCGAGAAGAAGG |
| | | TGAAGATGCTGGAGCAGCAGCGCAGTGAGCTGCTGGAAGTGAACAAGCAGTGGGACCAGC |
| | | ATTTCCGGTCCATGAAGCAGCAGTATGAGCAGAAGATCACTGAGCTGCGTCAGAAGCTGGC |
| | | TGATTTGCAGAAGCAGGTGACTGACCTGGAGGCCGAGCGGGAGCAGAAGCAGCGTGACTTT |
| | | GACCGCAAGCTCCTCCTGGCCAAGTCCAAGATTGAAATGGAGGAGACCGACAAGGAGCAG |
| | | CTGACAGCAGAGGCCAAGGAGCTGCGCCAAAAGGTCAAGTACCTGCAGGATCAGCTGAGC |
| | | CCACTCACCCGACAGCGTGAGTACCAGGAAAAGGAGATCCAGCGGCTCAACAAGGCCCTG |
| | | GAGGAAGCACTGAGCATCCAAACCCCGCCATCATCTCCACCAACAGCATTTGGGAGCCCAG |
| | | AAGGAGCAGGGGCCCTCCTAAGGAAACAGGAGCTGGTCACGCAGAATGAGTTGCTGAAAC |
| | | AGCAGGTGAAGATCTTCGAGGAGGACTTCCAGAGGGAGCGCAGTGATCGTGAGCGCATGA |
| | | ATGAGGAGAAGGAAGAGCTGAAGAAGCAAGTGGAGAAGCTGCAGGCCCAGGTCACCCTGT |
| | | CAAATGCCCAGCTAAAAGCATTCAAAGATGAGGAGAAGGCAAGAGAAGCCCTCAGACAGC |
| | | AGAAGAGGAAAGCAAAGGCCTCAGGAGAGCGTTACCATGTGGAGCCCCACCCAGAACATC |
| | | TCTGCGGGGCCTACCCCTACGCCTACCCGCCCATGCCAGCCATGGTGCCACACCATGGCTTC |
| | | GAGGACTGGTCCCAGATCCGCTACCCCCCTCCCCCCATGGCCATGGAGCACCCGCCCCCACT |
| | | CCCCAACTCGCGCCTCTTCCATCTGCCGGAATACACCTGGCGTCTACCCTGTGGAGGGGTTC |
| | | GAAATCCAAATCAGAGCTCCCAAGTGATGGACCCTCCCACAGCCAGGCCTACAGAACCAGA |
| | | GTCTCCAAAAAATGACCGTGAGGGGCCTCAGTNNCCAACTTTCTTGTACAAAGTTGGCATTA |
| | | TAAGAAAGCATTGCTTATCAATTTGTTGCAACGAAC |
| 35 | Homo | GTTCGTTGCAACAAATTGATGAGCAATGCTTTTTTATAATGCCAACTTTGTACAAAAAAGTT |
| | sapiens clone | GGCACCATGGCGGAGCTGCGCGTGCTCGTAGCTGTCAAGAGGGTCATCGACTACGCCGTGA |
| | CCSBHm_00036156 | AGATCCGAGTGAAGCCTGACAGGACCGGTGTGGTCACGGATGGTGTGAAGCACTCCATGAA |
| | ETFB | CCCCTTCTGTGAGATCGCGGTGGAGGAGGCTGTGCGGCTCAAGGAGAAGAAGCTGGTGAAG |
| | (ETFB) | GAGGTCATCGCCGTCAGCTGTGGGCCTGCACAGTGCCAGGAGACGATTCGTACCGCCCTGG |
| | mRNA. | CCATGGGTGCAGACCGAGGTATCCACGTGGAGGTGCCCCCAGCAGAAGCAGAACGCTTGGG |
| | GenBank: | TCCCCTGCAGGTGGCTCGGGTCCTGGCCAAGCTGGCAGAGAAGGAGAAGGTGGACCTGGTG |
| | KR712152.1 | CTGCTGGGCAAACAGGCCATCGATGATGACTGTAACCAGACAGGGCAGATGACAGCTGGAT |
| | | TTCTTGACTGGCCACAGGGCACATTCGCCTCCCAGGTGACGCTGGAGGGGGACAAGTTGAA |
| | | AGTGGAGTGGGAGATCGATGGGGGCCTGGAGACCCTGCGCCTGAAGCTGCCAGCTGTGGTG |
| | | ACAGCTGACCTGAGGCTCAACGAGCCCCGCTACGCCACGCTGCCCAACATCATGAAAGCCA |
| | | AGAAGAAGAAGATCGAGGTGATCAAGCCTGGGGACCTGGGTGTGGACCTGACCTCCAAGCT |
| | | CTCTGTGATCAGTGTGGAGGACCCGCCCCAGCGCACGGCCGGCGTCAAGGTGGAGACCACT |
| | | GAGGACCTGGTGGCCAAGCTGAAGGAGATTGGGCGGATTTTGCCAACTTTCTTGTACAAAG |
| | | TTGGCATTATAAGAAAGCATTGCTTATCAATTTGTTGCAACGAAC |
| 36 | Homo | GTTCGTTGCAACAAATTGATGAGCAATGCTTTTTTATAATGCCAACTTTGTACAAAAAAGTT |
| | sapiens clone | GGCATGGTTGGGTTCAAGGCCACAGATGTGCCCCCTACTGCCACTGTGAAGTTTCTTGGGGC |
| | CCSBHm_00012307 | TGGCACAGCTGCCTGCATCGCAGATCTCATCACCTTTCCTCTGGATACTGCTAAAGTCCGGT |
| | UCP2 | TACAGATCCAAGGAGAAAGTCAGGGGCCAGTGCGCGCTACAGCCAGCGCCCAGTACCGCG |
| | (UCP2) | GTGTGATGGGCACCATTCTGACCATGGTGCGTACTGAGGGCCCCCGAAGCCTCTACAATGG |
| | mRNA. | GCTGGTTGCCGGCCTGCAGCGCCAAATGAGCTTTGCCTCTGTCCGCATCGGCCTGTATGATT |
| | GenBank: | CTGTCAAACAGTTCTACACCAAGGGCTCTGAGCATGCCAGCATTGGGAGCCGCCTCCTAGC |
| | KR710423.1 | AGGCAGCACCACAGGTGCCCTGGCTGTGGCTGTGGCCCAGCCCACGGATGTGGTAAAGGTC |
| | | CGATTCCAAGCTCAGGCCCGGGCTGGAGGTGGTCGGAGATACCAAAGCACCGTCAATGCCT |
| | | ACAAGACCATTGCCCGAGAGGAAGGGTTCCGGGGCCTCTGGAAAGGGACCTCTCCCAATGT |
| | | TGCTCGTAATGCCATTGTCAACTGTGCTGAGCTGGTGACCTATGACCTCATCAAGGATGCCC |
| | | TCCTGAAAGCCAACCTCATGACAGATGACCTCCCTTGCCACTTCACTTCTGCCTTTGGGGCA |
| | | GGCTTCTGCACCACTGTCATCGCCTCCCCTGTAGACGTGGTCAAGACGAGATACATGAACTC |
| | | TGCCCTGGGCCAGTACAGTAGCGCTGGCCACTGTGCCCTTACCATGCTCCAGAAGGAGGGG |
| | | CCCCGAGGCTTCTACAAAGGGTTCATGCCCTCCTTTCTCCGCTTGGGTTCCTGGAACGTGGT |
| | | GATGTTCGTCACCTATGAGCAGCTGAAACGAGCCCTCATGGCTGCCTGCACTTCCCGAGAG |
| | | GCTCCCTTCTGCCCAACTTTCTTGTACAAAGTTGGCATTATAAGAAAGCATTGCTTATCAATT |
| | | TGTTGCAACGAAC |

In certain embodiments, the anti-CD47 antibody or antibody fragment thereof directly causes autonomous tumor cell death.

In certain embodiments, a method of monitoring the efficacy of a therapy for cancer in a patient undergoing the therapy is disclosed comprising, administering to the patient an anti-CD47 antibody or antibody binding fragment thereof, obtaining a biological sample from the patient and quantifying the amount of at least one biomarker in the biological sample, wherein the at least one biomarker is selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2;

quantifying the amounts of at least one biomarker in a baseline standard wherein the baseline standard is obtained from the patient prior to therapy; comparing the amounts of the at least one biomarker in the biological sample with the amount of at least one biomarker in the baseline standard; and determining that therapy is effective when the amount of at least one biomarker in the biological sample obtained from the patient is greater than the amounts of at least one biomarker present in the baseline standard.

In certain embodiments, the at least one biomarker is quantified on biological samples taken on two or more occasions from the patient.

In certain embodiments, the one of the two or more occasions is prior to commencement of therapy and one of the two or more occasions is after commencement of therapy.

In certain embodiments, the effect the therapy has on an individual is determined based a change in the amount of the biomarkers in biological samples taken on two or more occasions.

In certain embodiments, the biological samples are taken at intervals over the course of therapy with an anti-CD47 antibody.

In certain embodiments, a method is disclosed to determine increased tumor cell death or resistance in the presence of an anti-CD47 antibody, comprising: transfecting mRNA encoding an RNA-guided endonuclease into a tumor cell line, wherein the RNA-guided endonuclease is expressed from the transfected mRNA; introducing a DNA vector that encodes a specific guide RNA, wherein the specific guide RNA directs the RNA-guided endonuclease to at least one targeted locus in the tumor cell genome; cleaving the at least one targeted locus in the tumor cell genome with the RNA-guided endonuclease; generating a genetic modification at the site of the cleavage; expanding the resulting genetically modified tumor cells; treating genetically modified tumor cells with an anti-CD47 antibody; and assaying genetically modified tumor cells to determine if targeted gene locus is responsible for cell death or gene-mediated resistance.

In some embodiments, the tumor cell is a solid tumor or a hematological tumor. In some embodiments, the targeted locus is selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2.

### Checkpoints and Checkpoint Inhibition

Therapeutic antibodies targeting the T cell checkpoints, PD-1, PD-L1 and CTLA-4 to enhance the cytotoxic activity of the adaptive T cell immune response have raised the prospect of long-term remission or even cure for patients with metastatic diseases (Hodi et al., 2010; McDermott et al., 2015). Despite positive results, there remains a significant patient population that either fails to respond to these checkpoint inhibitors (primary resistance) or those that respond, but eventually develop disease progression (acquired resistance) (Pitt et al., 2016; Restifo et al., 2016; Sharma et al., 2017). Recent studies suggest that resistance mechanisms can be both tumor cell intrinsic, including a lack of unique tumor antigen proteins or inhibition of tumor antigen presentation, and tumor cell extrinsic, involving the absence of infiltrating T cells, redundant inhibitory checkpoints and/or the presence of immunosuppressive cells in the tumor microenvironment (Sharma et al., 2017). Even in tumors considered sensitive to checkpoint inhibitors, or when combining anti-CTLA-4 and anti-PD-1/PDL-1 agents, approximately 50% of patients do not experience tumor shrinkage and the median treatment duration or progression-free survival for all treated patients remains relatively short around 2-5 months (Kazandjian et al., 2016). In addition, several of the most prevalent solid tumors and the majority of hematological malignancies have shown disappointing results with these checkpoint inhibitors. In particular, hormone receptor-positive breast cancer, colorectal cancer (non-microsatellite instability) and prostate cancer do not appear to be sensitive to this type of immune manipulation and could benefit from a different immunotherapy approach (Le et al., 2015; Dirix et al., 2015; Topalian et al., 2012; Graff et al., 2016). These findings highlight the need for alternative or synergistic approaches that target additional checkpoints to activate the innate immune response in addition to the adaptive immune response to further improve clinical outcomes.

### CD47 Biology and Role as Innate Immune Checkpoint

CD47, also known as integrin associated protein (IAP), is a 50 kDa cell surface Ig superfamily member containing an extracellular IgV domain, 5 transmembrane domains, and a short C-terminal cytoplasmic tail, that is expressed on most cells and overexpressed on many cancer subtypes (Lindberg et al., 1993; Reinhold et al., 1995; Majeti et al., 2009; Willingham, 2012). The functional activities of CD47 are defined by its two distinct ligands, signal regulatory protein alpha (SIRPα) and thrombospondin 1 (TSP1) (Gao et al., 1994; Barclay et al., 2006). TSP is present on plasma and is synthesized by many cells, including platelets. SIRPα is expressed on many hematopoietic cells, including macrophages, dendritic cells, granulocytes and on a number of other cell types including neurons and glia. The CD47/SIRPα interaction functions as a marker of self, regulating macrophage and dendritic cell phagocytosis of target cells sending a “don't eat me signal” to the phagocyte. The binding of CD47 to SIRPα initiates an inhibitory signaling cascade resulting in inhibition of phagocytosis following phosphorylation of its cytoplasmic immunoreceptor tyrosine-based inhibition motifs (ITIMs) (Oldenborg et al., 2000; Oldenborg et al., 2001; Okazawa et al., 2005), recruitment and binding of SHP1 and SHP2, Src homology domain-containing protein tyrosine phosphatases (Veillette et al., 1998; Oldenborg et al., 2001), inhibition of non-muscle myosin IIA and ultimately phagocytic function (Tsai and Discher et al., 2008; Barclay and van den Berg, 2014; Murata et al., 2014; Veillette and Chen, 2018; Matazaki et al., 2009). Several anti-CD47 mouse antibodies have been described that block the interaction of CD47 and SIRPα, including B6H12 (Seiffert et al., 1999; Latour et al., 2001; Subramanian et al., 2006, Liu et al., 2002; Rebres et al., 2005), BRIC126 (Vernon-Wilson et al., 2000; Subramanian et al., 2006), CC2C6 [Seiffert et al., 1999) and 1F7 (Rebres et al., 2005). B6H12 and BRIC126 have also been shown to cause phagocytosis of human tumor cells by human and mouse macrophages (Willingham et al., 2012; Chao et al., 2012; EP 2242512 B1]. This increase in phagocytic activity resulting from blocking the CD47/SIRPα is the primary mechanism of action for the CD47 antibodies currently in early clinical studies (Barclay et al., 2014; Weiskopf et al., 2017). This mechanism serves as one of the most important checkpoints regulating innate immune activation. Anti-CD47 mAbs have also been shown to promote an adaptive immune response to tumors in vivo Tseng et al., 2013; Soto-Pantoja et al., 2014; Liu et al., 2015). Cancer cells thus use CD47 to mask themselves in “selfness” to evade both the innate and adaptive immune systems.

### Inducing Death of Tumor Cells

Some soluble anti-CD47 mAbs initiate a cell death program on binding to CD47 on tumor cells, resulting in collapse of mitochrondrial membrane potential, loss of ATP generating capacity, increased cell surface expression of phosphatidylserine (detected by increased staining for annexin V) and cell death without the participation of caspases or fragmentation of DNA. Such soluble anti-CD47 mAbs have the potential to treat a variety of solid and hematological cancers. Several soluble anti-CD47 mAbs which have been shown to induce tumor cell death, including MABL-1, MABL-2 and fragments thereof (U.S. Pat. No. 8,101,719; Uno et al. Oncol Rep. 17: 1189-94, 2007; Kikuchi et al. Biochem Biophys Res. Commun. 315: 912-8, 2004), Ad22 (Pettersen et al. J. Immuno. 166: 4931-4942, 2001; Lamy et al. J. Biol. Chem. 278: 23915-23921, 2003), and 1F7 (Manna et al. J. Immunol. 170: 3544-3553, 2003; Manna et al. Cancer Research, 64: 1026-1036, 2004). Some of the anti-CD47 mAbs of the disclosure described herein induce cell death of human tumor cells.

Induction of cell death refers to the ability of certain of the soluble anti-CD47 antibodies, murine antibodies, chimeric antibodies, humanized antibodies, or antigen-binding fragments thereof (and competing antibodies and antigen-binding fragments thereof) disclosed herein to kill cancer cells via a cell autonomous mechanism without participation of complement or other cells including, but not limited to, T cells, neutrophils, natural killer cells, macrophages, or dendritic cells.

The terms “inducing cell death” or “kills” or “killing” and the like, are used interchangeably herein and refers to the functional characteristics of the anti-CD47 Abs as described herein.

As used herein, the term “induces death of human tumor cells” refers to increased binding of annexin V (in the presence of calcium) and increased 7-aminoactinomycin D (7-AAD) or propidium iodide uptake in response to treatment with an anti-CD47 mAb. These features may be quantitated in three cell populations: annexin V positive (annexin V⁺), annexin V positive/7-AAD negative (annexin V⁺/7-AAD⁻) and annexin V positive/7-AAD positive (annexin V⁺/7-AAD⁻) by flow cytometry. Induction of cell death may be defined by a greater than 2-fold increase in each of the above cell populations in human tumor cells caused by soluble anti-CD47 mAb compared to the background obtained with the negative control antibody, (humanized, isotype-matched antibody) or untreated cells.

Another indicator of cell death is loss of mitochondrial function and membrane potential by the tumor cells as assayed by one of several available measures (potentiometric fluorescent dyes such as DiO-C6 or JC1 or formazan-based assays such as MTT or WST-1).

As used herein, the term “causes loss of mitochondrial membrane potential” refers to a statistically significant (p<0.05) decrease in mitochondrial membrane potential by a soluble anti-CD47 mAb compared to the background obtained with a negative control, humanized isotype-matched antibody or no treatment.

Among the present humanized or chimeric mAbs, those that induce cell death of human tumor cells cause increased annexin V binding similar to the findings reported for anti-CD47 mAbs Ad22 (Pettersen et al. J. Immunol. 166: 4931-4942, 2001; Lamy et al. J. Biol. Chem. 278: 23915-23921, 2003); 1F7 (Manna and Frazier J. Immunol. 170:3544-3553, 2003; Manna and Frazier Cancer Res. 64:1026-1036, 2004); and MABL-1 and 2 (U.S. Pat. No. 7,531,643 B2; U.S. Pat. No. 7,696,325 B2; U.S. Pat. No. 8,101,719 B2).

Cell viability assays are described in NCI/NIH guidance manual that describes numerous types of cell-based assays that can be used to assess induction of cell death caused by CD47 antibodies: “Cell Viability Assays”, Terry L Riss, PhD, Richard A Moravec, B S, Andrew L Niles, M S, Helene A Benink, PhD, Tracy J Worzella, M S, and Lisa Minor, PhD. Contributor Information, published May 1, 2013.

### Induction of Programmed Cell Death III (PCDIII)

A number of anti-CD47 antibodies (CD47 mAbs), including Ad22, 1F7, MABL-1, MABL-2, and CC2C6, indicated that some, but not all, soluble CD47 mAbs, as well as some additional immobilized CD47 mAbs, can directly elicit a type of cell death of multiple types of tumor cells that is characteristic programmed cell death III (PCDIII) (Lie, 1999; Manna, 2003: Manna et al., 2004; Mateo et al., 1999; Mateo et al., 2002; Uno et al., 2005; Bras et al., 2007; Martinez-Torres et al., 2015; Leclair et al., 2018). PCDIII is caspase-independent and includes cellular features such as production of reactive oxygen species (ROS), loss of mitochondrial membrane potential (ΔΨₘ) and exposure of phosphatidylserine (PS) on the plasma membrane without the interaction with any immune effector cell (cell autonomous) and without nuclear features including chromatin condensation, DNA fragmentation and degradation (Kikuchi et al., 2005; Pettersen et al., 1999; Manna et al., 2003; Manna et al., 2004; Sagawa et al., 2011; Uno et al., 2007; Mateo et al., 1999; Mateo et al., 2002; Roue et al., 2003]. It is noteworthy that the anti-CD47 antibodies which induce cell death do not kill resting leukocytes, which also express CD47, but only those cells that are “activated” by transformation. Thus, normal circulating cells, many of which express CD47, are spared while cancer cells are selectively killed by the CD47 antibodies that possess this direct killing activity (Manna and Frazier, 2003).

### Induction of Immunogenic Cell Death (ICD)

The concept of immunogenic cell death (ICD) has emerged in recent years. This form of cell death, unlike non-immunogenic cell death, stimulates an adaptive immune response against tumor antigens presented to T cells (Casares, 2005; Krysko, 2012; Kroemer, 2012). ICD is induced by specific chemotherapy drugs, including anthracyclines (doxorubicin, daurorubicin and mitoxantrone) and oxaliplatin, but not by cisplatin and other chemotherapy drugs. ICD is also induced by bortezomib, cardiac glycosides, photodynamic therapy and radiation (Galluzi, 2016). ICD is characterized by the release or surface exposure of damage-associated molecular patterns (DAMPs) from dying cells that function as adjuvants for the immune system (Kroemer 2013). The distinctive characteristics of ICD of tumor cells are the release from or exposure on tumor cell surfaces these DAMPs including: 1) the pre-apoptotic cell surface exposure of calreticulin, 2) the secretion of adenosine triphosphate (ATP), 3) release of high mobility group box 1 (HMGB1), 4) annexin A1 release, 5) type I interferon release and 6) C-X-C motif chemokine ligand 10 (CXCL10) release. These ligands are endogenous damage-associated molecular patterns (DAMPs), which include the cell death-associated molecules (CDAMs). Importantly, each of these ligands induced during ICD binds to specific receptors, referred to as pattern recognition receptors (PRRs), that contribute to an anti-tumor immune response. ATP binds the purinergic receptors PY2, G-protein coupled, 2 (P2RY2) and PX2, ligand-gated ion channel, 7 (P2RX7) on dendritic cells causing dendritic cell recruitment and activation, respectively. Annexin A1 binds to formyl peptide receptor 1 (FPR1) on dendritic cells causing dendritic cell homing. Calreticulin expressed on the surface of tumor cells binds to LRP1 (CD91) on dendritic cells promoting antigen uptake by dendritic cells. HMGB1 binds to toll-like receptor 4 (TLR4) on dendritic cells to cause dendritic cell maturation. As a component of ICD, tumor cells release type I interferon leading to signaling via the type I interferon receptor and the release of the CXCL10 which favors the recruitment of effector CXCR3+ T cells Together, the actions of these ligands on their receptors facilitate recruitment of DCs into the tumor, the engulfment of tumor antigens by DCs and optimal antigen presentation to T cells. Kroemer et al. have proposed that a precise combination of the CDAMs mentioned above elicited by ICD can overcome the mechanisms that normally prevent the activation of anti-tumor immune responses (Kroemer et al., 2016). When mouse tumor cells treated in vitro with ICD-inducing modalities are administered in vivo to syngeneic mice, they provide effective vaccination that leads to an anti-tumor adaptive immune response, including memory. This vaccination effect cannot be tested in xenograft tumor models because the mice used in these studies lack a complete immune system. The available data indicate that ICD effects induced by chemotherapy or radiation will promote an adaptive anti-tumor immune response in cancer patients. The components of ICD are described in more detail below.

In 2005, it was reported that tumor cells which were dying in response to anthracycline chemotherapy in vitro caused an effective anti-tumor immune response when administered in vivo in the absence of adjuvant (Casares, et al. 2005). This immune response protected mice from subsequent re-challenge with viable cells of the same tumor and caused regression of established tumors. Anthracyclines (doxorubicin, daunorubicin and idarubicin) and mitomycin C induced tumor cell apoptosis with caspase activation, but only apoptosis induced by anthracyclines resulted in immunogenic cell death. Caspase inhibition did not inhibit cell death induced by doxorubicin but did suppress the immunogenicity of tumor cells dying in response to doxorubicin. The central roles of dendritic cells and CD8+ T cells in the immune response elicited by doxorubicin-treated apoptotic tumor cells was established by the demonstration that depletion of these cells abolished the immune response in vivo. Calreticulin is one of the most abundant proteins in the endoplasmic reticulum (ER). Calreticulin was shown to rapidly translocate pre-apoptotically from the ER lumen to the surface of cancer cells in response to multiple ICD inducers, including anthracyclines (Obeid et al., 2007, Kroemer et al., 2013). Blockade or knockdown of calreticulin suppressed the phagocytosis of anthracycline-treated tumor cells by dendritic cells and abolished their immunogenicity in mice. The exposure of calreticulin caused by anthracyclines or oxaliplatin is activated by an ER stress response that involves the phosphorylation of the eukaryotic translation initiation factor eIF2a by the PKR-like ER kinase. Calreticulin, which has a prominent function as an “eat-me” signal (Gardai, et al. 2005) binds to LRP1 (CD91) on dendritic cells and macrophages resulting in phagocytosis of the calreticulin expressing cell, unless the calreticulin-expressing cell expresses a don't eat me signal, such as CD47. Calreticulin also signals through CD91 on antigen presenting cells to cause the release of proinflammatory cytokines and to program Th17 cell responses. In summary, calreticulin expressed as part of ICD stimulates antigen presenting cells to engulf dying cells, process their antigens and prime an immune response.

In addition to calreticulin, protein disulfide-isomerase A3 (PDIA3), also called Erp57, was shown to translocate from the ER to the surface of tumor cells following treatment with mitoxantrone, oxaliplatin and irradiation with UVC light (Panaretakis et al., 2008; Panaretakis et al., 2009). A human ovarian cancer cell line, primary ovarian cancer cells and a human prostate cancer cell line expressed cell-surface calreticulin, HSP70 and HSP90 following treatment with the anthracyclines doxorubicin and idarrubicin (Fucikova et al., 2011). HSP70 and HSP90 bind to the PRR LRP1 on antigen presenting cells; the PRR to which PDIA3 binds has not been identified (Galluzi et al., 2016). TLR4 was shown to be required for cross-presentation of dying tumor cells and to control tumor antigen processing and presentation. Among proteins that were known to bind to and stimulate TLR4, HMGB1 was uniquely released by mouse tumor cells in which ICD was induced by irradiation or doxorubicin (Apetoh et al., 2007). The highly efficient induction of an in vivo anti-tumor immune by doxorubicin treatment of mouse tumor cells required the presence of HMGB1 and TLR4, as demonstrated by abrogation of the immune response by inhibition of HMGB1 and knock-out TLR4. These preclinical findings are clinically relevant. Patients with breast cancer who carry a TLR4 loss-of-function allele relapse more quickly after radiotherapy and chemotherapy than those carrying the normal TLR4 allele.

Ghiringhelli et al. showed that mouse tumor cells treated with oxaliplatin, doxorubicin and mitoxantrone in vitro released ATP and that the ATP binds to the purinergic receptors PY2, G-protein coupled, 2 (P2RY2) and PX2, ligand-gated ion channel, 7 (P2RX7) on dendritic cells (Ghiringhelli, et al., 2009). Binding of ATP to P2RX7 on DCs triggers the NOD-like receptor family, pyrin domain containing—3 protein (NLRP3)-dependent caspase-1 activation complex (inflammasome), allowing for the secretion of interleukin-β (IL-1β), which is essential for the priming of interferon-gamma-producing CD8+ T cells by dying tumor cells. Therefore, the ATP-elicited production of IL-1β by DCs appears to be one of the critical factors for the immune system to perceive cell death induced by certain chemotherapy drugs as immunogenic. This paper also reports that HMGB1, at TLR4 agonist, also contributes to the stimulation of the NLRP3 inflammasome in DCs and the secretion of IL-1β. These preclinical results have been shown to have clinical relevance; in a breast cancer cohort, the presence of the P2RX7 loss-of-function allele had a significant negative prognostic impact of metastatic disease-free survival. ATP binding to P2RY2 causes the recruitment of myeloid cells into the tumor microenvironment (Vacchelli et al., 2016).Michaud et al. demonstrated that autophagy is required for the immunogenicity of chemotherapy-induced cell death (Michaud, et al. 2011). Release of ATP from dying tumor cells required autophagy and autophagy-competent, but not autophagy-deficient, mouse tumors attracted dendritic cells and T lymphocytes into the tumor microenvironment in response to chemotherapy that induces ICD.

Ma et al. addressed the question of how chemotherapy-induced cell death leads to efficient antigen presentation to T cells (Ma et al., 2013). They found that at specific kind of tumor infiltrating lymphocyte, CD11c+CD11b+Ly6C^{hi} cells, are particularly important for the induction of anticancer immune responses by anthracyclines. ATP released by dying cancer cells recruited myeloid cells into tumors and stimulated the local differentiation of CD11c⁺CD11b⁺Ly6C^{hi} cells. These cells were shown to be particularly efficient in capturing and presenting tumor cell antigens and, after adoptive transfer into naïve mice, conferring protection to challenge with living tumor cells of the same cell line. It has been shown that anthracyclines stimulate the rapid production of type I interferons by tumor cells after activation of TLR3 (Sistugu et al., 2014). Type I interferons bind to IFNγ and IFNγ receptors on cancer cells and trigger autocrine and paracrine signaling pathways that result in release of CXCL10. Tumors lacking TLR3 or Ifnar failed to respond to chemotherapy unless type I IFN or CXCL10, respectively, was supplied. These preclinical findings have clinical relevance. A type I IFN-related gene expression signature predicted clinical responses to anthracycline-based chemotherapy in independent cohorts of breast cancer patients.

Another receptor on dendritic cells that is involved in chemotherapy-induced anticancer immune response was recently identified: formyl peptide receptor-1, which binds annexin A1 (Vacchelli et al., 2015). Vacchelli et al designed a screen to identify candidate genetic defects that negatively affect responses to chemotherapy. They identified a loss-of-function allele of the gene encoding formyl peptide receptor 1 (FPR1) that was associated with poor metastatis-free survival and overall survival in breast and colorectal cancer patients receiving adjuvant chemotherapy. The therapeutic effects of anthracyclines were abrogated in tumor-bearing Fpr1−/− mice due to impaired antitumor immunity. FPR1-deficient DCs did not approach dying tumor cells and, therefore, could not elicit antitumor T cell immunity. Two anthracyclines, doxorubicin and mitoxantrone, stimulated the secretion of annexin A1, one of four known ligands of FPR1. FPR1 and annexin A1 promoted stable interactions between dying cancer cells and human or mouse leukocytes.

In addition to anthracyclines and oxaliplatin, other drugs have been shown to induce immunogenic cell death. Cardiac glycosides, including clinically used digoxin and digitoxin, were also shown to be efficient inducers of immunogenic cell death of tumor cells (Menger et al., 2012). Other chemotherapy agents and cancer drugs that have been reported to induce DAMP expression or release are bleomycin, bortezomib, cyclophosphamide, paclitaxel, vorinistat and cisplatin (Garg et al., 2015, Menger et al., 2012, Martins et al., 2011). Importantly, these results have clinical relevance. Administration of digoxin during chemotherapy had a significant positive impact on the overall survival of patients with breast, colorectal, head and neck, and hepatocellular cancers, but failed to improve overall survival of lung and prostate cancer patients.

The anti-CD20 monoclonal antibody rituximab has improved outcomes in multiple B-cell malignancies. The success of rituximab, referred to as a type I anti-CD20 mAb, led to the development of type II anti-CD20 mAbs, including obinutuzumab and tositumomab. Cheadle et al investigated the induction of immunogenic cell death by anti-CD20 mAbs (Cheadle et al., 2013). They found that the cell death induced by obinutuzumab and tositumomab is a form of immunogenic cell death characterized by the release of HMGB1, HSP90 and ATP. A type I anti-CD20 mAb did not cause release of HMGB1, HSP90 and ATP. Incubation of supernatants from a human tumor cell line treated with obinutuzumab caused maturation of human dendritic cells, consistent with the previously described effects of HMGB1 and ATP on dendritic cells. In contrast to the results reported by Cheadle et al, Zhao et al reported that both type I and II anti-CD20 mAbs increased HMGB1 release from human diffuse large B cell lymphoma cell lines, but did not cause ATP release or cell surface expression of calreticulin [Zhao 2015]. A number of anti-CD47 mAbs have been shown to cause release from or exposure on tumor cell surfaces of the DAMPs listed above, characteristics of ICD alone and in combination with certain chemotherapeutic agents (WO Publications 2018/175790 and 2017/049251; and US Patent Publication 2018/0142019). These same antibodies also cause cell death characteristic of PCDIII.

Gene Expression Associated with PCDIII and ICD

The anti-CD47 antibodies which induce cell death characteristic of both PCDIII and ICD/DAMP induction whereby tumor cells undergo mitochondrial depolarization, increased production of ROS and phosphatidylserine exposure while at the same time exhibiting the surface exposure and release of DAMPs, results in significant and unique changes in gene expression. The tumor cells undergoing CD47 antibody induced PCDIII/ICD display significant changes in expression of genes that involved pathways related to ER stress, autophagy and JAK/STAT signaling, indicative of a cell experiencing critical organelle stress. These changes can be used as biomarkers to determine either responsiveness to treatment with these antibodies ex vivo prior to treatment of the patient or to determine responsiveness to treatment.

### Synthetic Lethality

As described herein, synthetic lethality is defined as the simultaneous disruption of two or more genes (Gene 1 and Gene 2, and Gene 3, Gene 4 . . . , etc.) which can cause cell death, whereas the disruption of either gene (Gene 1 or Gene 2) in isolation does not result in cell death. This concept has been leveraged in gene knockout screens to uncover novel genes whose inactivation causes a lethal phenotype which is dependent on the mutations already present in a tumor cell (Wang et al. 2014, Zhou et al 2014). Current gene loss-of-function screens are commonly carried out by utilizing the Clustered Regularly Interspaced Short Palindromic Repeats (CRISPR)/Cas9 system. CRISPR/Cas9 is a two-component technology, consisting of single guide RNAs (sgRNA) and the Cas9 DNA nuclease. The sgRNA acts as a targeting agent, directing cleavage of genomic DNA by Cas9. These DNA cleavage events trigger mistake-prone DNA repair pathways, which frequently produce insertions or deletions into the DNA sequence. This causes frameshifting in the gene protein-coding regions, which often results in complete loss-of-function of the gene. CRISPR/Cas9 technology can be applied in high-throughput screening formats in which thousands of sgRNAs (the “library”) are combined into a cell population all at once (‘pooled format”). Pooled screening quantitatively compares the relative abundance of individual sgRNAs in the population before and after prolonged culture, either in the absence or presence of (drug) selection. Those sgRNAs targeting genes that upon inactivation cause a lethal phenotype will be reduced in the population over time. The sgRNAs targeting genes that upon inactivation increase tumor cell survival in the presence of drug selection will increase in the population over time. In this latter case, mediators of resistance to therapy can be identified. The abundance of each individual gRNA can be determined by using high-throughput sequencing of genomic DNA, and the fold change or depletion can be determined among the different populations.

### Definitions

As used herein, the term “patient” as used herein refers to a human, for whom a classification as a responder to a next generation immune checkpoint inhibitor is desired, and for whom further treatment can be provided.

As used herein, the term “the baseline standard” is a unit of measurement which allows for calibration of the biological effects which may occur after the administration of a therapy; i.e. an anti-CD47 antibody or antigen binding fragment thereof which induces tumor cell death.

As used herein, the term “biomarker” is a biological molecule found in blood, other body fluids, or tissues that is a sign of a normal or abnormal process, a condition, or disease. A biomarker is a characteristic that is objectively measured and evaluated as an indicator of normal biological processes, pathogenic processes or pharmacologic responses to a therapeutic intervention, e.g., the administration of an anti-CD47 antibody or antigen binding fragment thereof. The term “biomarker” can be used interchangeably with molecular marker and/or signature molecule and may be categorized as DNA biomarkers, DNA tumor biomarkers, protein biomarkers, and other general biomarkers.

As used herein, the terms “tumor” or “tumor tissue” refer to an abnormal mass of tissue that results from excessive cell division. A tumor or tumor tissue comprises “tumor cells” which are neoplastic cells with abnormal growth properties and no useful bodily function. Tumors, tumor tissue and tumor cells may be benign or malignant. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “malignancy” refers to a non-benign tumor or a cancer. As used herein, the term “cancer” connotes a type of hyperproliferative disease which includes a malignancy characterized by deregulated or uncontrolled cell growth.

The following examples are included to demonstrate preferred embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### EXAMPLES

### Example 1

### NanoString Gene Expression Profiling of Human Leukemia Cells Undergoing Anti-CD47 Induced Cell Death (PCDIII/ICD)

NanoString gene expression profiling was performed on a purified population of cells that were actively undergoing anti-CD47-mediated cell death as determined by PS exposure. Jurkat human leukemia cells were treated with 10 μg/ml of an anti-CD47 antibody which induces tumor cell death as disclosed herein, an anti-CD47 antibody that does not induce tumor cell death, Hu-5F9, and an IgG2 antibody (control). Cells treated with an anti-CD47 antibody that induce tumor cell death were sorted with Annexin V conjugated beads to enrich for cells undergoing cell death. Cells treated with IgG2 Ab or Hu-5F9 or an anti-CD47 Ab that does not induce tumor cell death were not sorted. RNA was harvested from Annexin V+ enriched cells, which were treated with an anti-CD47 Ab that does induce tumor cell death and from control cells treated with IgG2 Ab and Hu-5F9 at selected timepoints between 2 hours and 48 hours. The RNA was submitted for NanoString gene expression profiling. A custom NanoString gene expression profiling panel was designed based on a previously obtained RNAseq dataset from an anti-CD47 Ab which induces cell death.

As shown in FIG. 1A, the gene set enrichment analysis data demonstrated that a treatment with an anti-CD47 tumor cell death inducing antibody upregulated genes that were distinct from control cells or cells treated with an anti-CD47 antibody that does not induce tumor cell death (Hu5F9). The genes that were upregulated with an anti-CD47 Ab that induces tumor cell death antibody were analyzed by NanoString gene expression profiling and were grouped into functional pathways as shown in FIG. 1B. Pathways know to be involved in CD47-mediated cell death which are represented include ER stress/Unfolded Protein Response, phosphatidylserine exposure/apoptosis, cAMP/PKA, and mitochondrial stress, all of which are consistent with PCDIII. Pathways identified which are consistent with ICD include ATP and HMGB1 secretion and autophagy and JAK/STAT signaling.

### Example 2

### Synthetic Lethal Targeting of CD47 Signaling Pathways

As described herein, a CRISPR/Cas9-based gene knockout screen will be used to identify genes by which inactivation will lead to either increased tumor cell death (synthetic lethality) or will lead to increased resistance to cell death (mediators of resistance) in the presence of anti-CD47 antibody therapy. To carry out a whole genome CRISPR/Cas9 gene knockout screen in the presence of anti-CD47 therapy, human tumor cell lines will be generated to express Cas9 nuclease using a lentiviral based expression system. Next, a library of sgRNAs in recombinant lentiviral vectors will be used to infect the Cas9-expressing tumor cells. Successfully transduced cells will be selected either by monitoring co-expression of fluorescent proteins, or antibiotic resistance. Once sgRNA-bearing cells are selected, they will be split into two treatment groups, either control IgG antibody in solution or anti-CD47 antibody in soluble form. The sgRNA-library harboring cells will be cultured in the presence of antibody for at least 14 days and genomic DNA isolated and analyzed by next generation sequencing to determine sgRNA abundance before and after antibody treatment. Validation of single gene synthetic lethality or resistance when in combination with CD47 antibody treatment will be determined by generation of individual sgRNA lentiviral vectors and transduction into Cas9-expressing tumor cells followed by treatment with control or anti-CD47 antibody. If cell death increases above baseline in the anti-CD47 group, but not in the IgG control antibody group, this would be evidence of a synthetic lethal interaction. If cell death decreases below baseline levels in the anti-CD47 group, but not in the IgG control antibody group, this would be evidence of a gene mediating resistance to anti-CD47 therapy.

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

Also provided are embodiments wherein any embodiment above can be combined with any one or more of these embodiments, provided the combination is not mutually exclusive. Also provided herein are uses in the treatment of indications or one or more symptoms thereof as disclosed herein and uses in the manufacture of medicaments for the treatment of indications or one or more symptoms thereof as disclosed herein, equivalent in scope to any embodiment disclosed above, or any combination thereof that is not mutually exclusive.

1. A method of treating of cancer in a patient with at least one biomarker having an amount greater than the amount of a baseline standard comprising: administering an effective amount of an anti-CD47 antibody to the patient, wherein at least one biomarker is selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2.

2. A method of diagnosing and treating a cancer in a patient who has not received therapy, comprising:
a. obtaining a biological sample from a patient and administering an anti-CD47 antibody to the biological sample in vitro;
b. quantifying the amount of at least one biomarker:
i. in said biological sample treated with an anti-CD47 antibody, and
ii. in an untreated biological sample,
wherein the at least one biomarker is selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2;
c. comparing the amounts of the at least one biomarker in step b.i. with the biological sample in step b.ii;
d. identifying the patient as being responsive to anti-CD47 therapy when the amount of at least one biomarker in the biological sample is greater than the amount of at least one biomarker in the untreated biological sample from the patient; and
e. administering a therapeutically effective amount of an anti-CD47 antibody to the patient.

3. The method of claim 2, wherein the quantifying is performed by measuring the amount of the biomarker in each sample by one or more methods selected from NanoString gene expression profiling, RNAseq, qPCR, and microarray.

4. The method of claim 1 or 2, wherein the cancer is a solid tumor, leukemia, a lymphoma, sarcoma, or multiple myeloma.

5. The method of claim 4, wherein the solid tumor is selected from ovarian cancer, breast cancer, endometrial cancer, colon cancer (colorectal cancer), rectal cancer, bladder cancer, urothelial cancer, lung cancer (non-small cell lung cancer, adenocarcinoma of the lung, squamous cell carcinoma of the lung), bronchial cancer, bone cancer, prostate cancer, pancreatic cancer, gastric cancer, adrenocortical carcinoma, hepatocellular carcinoma, adult (primary) liver cancer, gall bladder cancer, bile duct cancer, esophageal cancer, renal cell carcinoma, thyroid cancer, squamous cell carcinoma of the head and neck (head and neck cancer), testicular cancer, cancer of the endocrine gland, cancer of the adrenal gland, cancer of the pituitary gland, cancer of the skin, cancer of soft tissues, cancer of blood vessels, cancer of brain, cancer of nerves, cancer of eyes, cancer of meninges, cancer of oropharynx, cancer of hypopharynx, cancer of cervix, and cancer of uterus, glioblastoma, meduloblastoma, astrocytoma, glioma, meningioma, gastrinoma, neuroblastoma, melanoma, and myelodysplastic syndrome.

6. The method of claim 4 wherein the leukemia is selected from systemic mastocytosis, acute lymphocytic (lymphoblastic) leukemia (ALL), T-cell—ALL, acute myeloid leukemia (AML), chronic lymphocytic leukemia (CLL), chronic myeloid leukemia (CML), myeloproliferative disorder/neoplasm, myelodysplastic syndrome, monocytic cell leukemia, and plasma cell leukemia.

7. The method of claim 4 wherein the lymphoma is selected from histiocytic lymphoma and T-cell lymphoma, B cell lymphomas, including Hodgkin's lymphoma and non-Hodgkin's lymphoma, such as low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, and Waldenstrom's Macroglobulinemia.

8. The method of claim 4, wherein the sarcoma is selected from osteosarcoma, Ewing's sarcoma, leiomyosarcoma, synovial sarcoma, alveolar soft part sarcoma, angiosarcoma, liposarcoma, fibrosarcoma, rhabdomyosarcoma, and chrondrosarcoma

9. The method of claim 1 or 2, wherein the biological sample is a core biopsy, free needle aspirate, pleural effusion, resection, ascites, whole blood, blood serum, plasma, bone marrow, or other bodily fluid, or dilution thereof.

10. The method of claim 1 or 2 wherein at least two biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

11. The method of claim 1 or 2 wherein at least three biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

12. The method of claim 1 or 2 wherein at least four biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

13. The method of claim 1 or 2 wherein at least five biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

14. The method of claim 1 or 2 wherein at greater that five biomarkers are selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.

15. The method of claim 1 or 2, wherein the anti-CD47 antibody directly causes autonomous tumor cell death.

16. The method of claim 1 or 2, wherein the anti-CD47 antibody is selected from combination of a heavy chain (HC) and a light chain (LC), wherein the combination is selected from:
a heavy chain comprising the amino acid sequence of SEQ ID NO:1 and a light chain comprising the amino acid sequence SEQ ID NO:2;
a heavy chain comprising the amino acid sequence of SEQ ID NO:3 and a light chain comprising the amino acid sequence SEQ ID NO:4;
a heavy chain comprising the amino acid sequence of SEQ ID NO:5 and a light chain comprising the amino acid sequence SEQ ID NO:6;
a heavy chain comprising the amino acid sequence of SEQ ID NO:7 and a light chain comprising the amino acid sequence SEQ ID NO:6;
a heavy chain comprising the amino acid sequence of SEQ ID NO:8 and a light chain comprising the amino acid sequence SEQ ID NO:9; and
a heavy chain comprising the amino acid sequence of SEQ ID NO:7 and a light chain comprising the amino acid sequence SEQ ID NO:10.

17. A method of monitoring efficacy of a therapy for cancer in a patient undergoing the therapy, comprising:
a. administering to the patient an anti-CD47 antibody or antigen binding fragment thereof,
b. obtaining a biological sample from the patient and quantifying the amount of at least one biomarker in the biological sample, wherein the at least one biomarker is selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof;
c. quantifying the amounts of at least one biomarker in a baseline standard wherein the baseline standard is obtained from the patient prior to therapy;
d. comparing the amounts of the at least one biomarker in the biological sample with the amount of at least one biomarker in the baseline standard; and
e. determining that therapy is effective when the amount of at least one biomarker in the biological sample obtained from the patient is greater than the amounts of at least one biomarker present in the baseline standard.

18. The method of claim 17, wherein the at least one biomarker is quantified on biological samples taken on two or more occasions from the patient.

19. The method of claim 18, wherein one of the two or more occasions is prior to commencement of therapy and one of the two or more occasions is after commencement of therapy.

20. The method of claim 17, wherein an effect the therapy has on an individual is determined based a change in the amount of the biomarkers in the biological samples taken on two or more occasions.

21. The method of claim 17, wherein the biological samples are taken at intervals over the course of therapy with an anti-CD47 antibody or antigen binding fragment thereof.

22. A method to determine increased tumor cell death in the presence of an anti-CD47 antibody, comprising:
a. transfecting mRNA encoding an RNA-guided endonuclease into a tumor cell line, wherein the RNA-guided endonuclease is expressed from the transfected mRNA;
b. introducing a DNA vector that encodes a specific guide RNA, wherein the specific guide RNA directs the RNA-guided endonuclease to at least one targeted locus in the tumor cell genome;
c. cleaving the at least one targeted locus in the tumor cell genome with the RNA-guided endonuclease;
d. generating a genetic modification at the site of the cleavage; expanding the resulting genetically modified tumor cells;
e. treating genetically modified tumor cells with an anti-CD47 antibody or antigen binding fragment thereof; and
f. assaying genetically modified tumor cells to determine if targeted gene locus is responsible for cell death or gene-mediated resistance.

23. The method of claim 22, wherein the tumor cell is a solid tumor or a hematological tumor.

24. The method of claim 22, wherein the targeted locus is selected from XBP1, PPP1R15A, UBR4, TRIB3, GYS1, PCK2, SCAP, SREBF1, RAPGEF1, TLN1, PARD6A, RND1, XKR8, ANO6, SLC26A6, PTPN4, ATG9A, MAP1LC3B, AMBRA1, MUL1, STAT3, IFNAR2, STAT5A, TNIP1, ETFB, UCP2 or variants thereof.
